SEQUENCE LISTING (20 sequences)

SEQ ID NO: 1 - DNA, 22 bp, Artificial Sequence
  synthetic oligonucleotide primer
cgttctctttggaaaggtgttc

SEQ ID NO: 2 - DNA, 20 bp, Artificial Sequence
  synthetic oligonucleotide primer
acactcggaccacgtctttc

SEQ ID NO: 3 - DNA, 20 bp, Artificial Sequence
  synthetic oligonucleotide primer
cgcaccactggcattgtcat

SEQ ID NO: 4 - DNA, 20 bp, Artificial Sequence
  synthetic oligonucleotide primer
ttctccttgatgtcacgcac

SEQ ID NO: 5 - DNA, 20 bp, Artificial Sequence
  synthetic oligonucleotide primer
cagaacctgctgcctgaatg

SEQ ID NO: 6 - DNA, 21 bp, Artificial Sequence
  synthetic oligonucleotide primer
gtagaaatgcctgaggaaacg

SEQ ID NO: 7 - DNA, 20 bp, Artificial Sequence
  synthetic oligonucleotide primer
gagtttacaggcttgtggca

SEQ ID NO: 8 - DNA, 20 bp, Artificial Sequence
  synthetic oligonucleotide primer
gagggcaattcctgaggatt

SEQ ID NO: 9 - DNA, 21 bp, Artificial Sequence
  synthetic oligonucleotide primer
ccatgtacatgagcactgttg

SEQ ID NO: 10 - DNA, 21 bp, Artificial Sequence
  synthetic oligonucleotide primer
ctccaataactcctggtatcc

SEQ ID NO: 11 - DNA, 20 bp, Artificial Sequence
  synthetic oligonucleotide primer
cgcacggaatttgaacagta

SEQ ID NO: 12 - DNA, 20 bp, Artificial Sequence
  synthetic oligonucleotide primer
ggatcagggacctgtcacac

SEQ ID NO: 13 - DNA, 21 bp, Artificial Sequence
  synthetic oligonucleotide primer
cagctggcgcacctcaagatg

SEQ ID NO: 14 - DNA, 23 bp, Artificial Sequence
  synthetic oligonucleotide primer
agggaagttgggctcaggactgg

SEQ ID NO: 15 - DNA, 23 bp, Artificial Sequence
  synthetic oligonucleotide primer
aacagacacagccctcacaaaca

SEQ ID NO: 16 - DNA, 23 bp, Artificial Sequence
  synthetic oligonucleotide primer
cgggaacttgaactggaactgac

SEQ ID NO: 17 - DNA, 20 bp, Artificial Sequence
  synthetic oligonucleotide primer
ttgcaggcactcagctagaa

SEQ ID NO: 18 - DNA, 20 bp, Artificial Sequence
  synthetic oligonucleotide primer
tgttcatggacagtggcatt

SEQ ID NO: 19 - DNA, 20 bp, Artificial Sequence
  synthetic oligonucleotide primer
ctgacctcacctgggacaat

SEQ ID NO: 20 - DNA, 20 bp, Artificial Sequence
  synthetic oligonucleotide primer
ccatcaaggcacagcaactc

(19) United States
(12) Patent Application Publication
(10) Pub. No.: US 2005/0095708 A1
(43) Pub. Date: May 5, 2005

(54) Characterization and isolation of subsets of human embryonic stem cells (HES) and cells associated or derived therefrom

(76) Inventors: Martin Frederick Pera, Prahran (AU); Andrew Laslett, Clayton (AU); Susan Melanie Hawes, Clayton (AU); Tomonobu Gion, Clayton (AU)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/841,907

(22) Filed: May 7, 2004

Related U.S. Application Data
(63) Continuation of application No. PCT/AU02/01534, filed on Nov. 11, 2002.

(30) Foreign Application Priority Data
Nov. 9, 2001 (AU) PR8781
Mar. 15, 2002 (AU) PS1129

(51) Int. Cl. C12N 5/08

(52) U.S. Cl. 435/369

(57) ABSTRACT

A sub-population of HES cells is identified which is positive for specific HES markers. These cell types are identified by expression of particular GCTM-2 antigens. A method based on the identification of GCTM-2 can differentiate between substantially undifferentiated or differentiated cell populations. Spontaneous differentiation often occurs in HES cultures resulting in non-homogeneous cell cultures. The invention also provides further sub populations of cells within differentiated cultures of HES cells that are SCL or epithelial in nature.

The present invention relates to methods of isolating sub-populations of viable Human Embryonic Stem Cells (HES) in particular to distinguish between pluripotent and differentiated cells in a HES population and to provide cell populations comprising substantially pure populations of the same cells in either a pluripotent or differentiated form. The invention also provides distinct cell populations derived from the pluripotent or differentiated cell populations as well as progeny derived therefrom.

### BACKGROUND

Human embryonic stem (HES) cells are cultured cell lines derived from the inner cell mass of the human blastocyst (Pera et al., 2000). HES cells may be serially cultivated indefinitely in the primitive embryonic state whilst retaining the key property of the cells from which they were derived: this being pluripotentiality or the ability to differentiate into all the cells of the adult body. These two features make human embryonic stem cells a potentially powerful technology platform for research into human health and disease, and a source of cells for therapeutic application in the field of regenerative medicine. In order to fulfil the potential of HES cells in research and therapy, it is desirable to develop the means to culture HES cells in a pure form on a large scale, and to obtain pure populations of specific types of differentiated or pluripotent cells from them. In particular in the fields of gastroenterology, diabetes, and respiratory medicine, the ability to isolate and cultivate the endodermal precursors which give rise to gut, liver, pancreas and lung, would be a very significant advance.

Several attempts to obtain pure populations of ES cells have not resulted in suitably homogeneous populations with the result that insufficient cell numbers retained in a pluripotent state have not been ideal for satisfying the requirements for research and therapy. Furthermore, pure populations of cells which are not dependent upon feeder layers would be desirable particularly for the purposes of therapy as the presence of feeder layers particularly of non-human origin would be most desirable to avoid any cross contamination.

Recent attempts to refine the basic cell culture system and apply it to culturing HES have still required feeder cell layers. Advances using growth factors and varying conditions or cell culturing have not drastically improved HES cell cultivation and serial cultivation of human ES cells and derivation of pure populations of differentiated cells from them, in particular, cells of endodermal origin, remains problematic. The HES cell cultures described to date are not homogenous but contain a mixture of different cell types. Spontaneous differentiation, a process which is constantly ongoing in ES cell cultures, gives rise to cells which can inhibit stem cell renewal. Therefore further characterisation of the stem cell populations and the development of methods to purify stem cells in a viable form are required. Present methods of obtaining differentiated cells, which rely on spontaneous differentiation followed by selection, often fail to produce adequate numbers of desired end cells in a sufficiently pure form.

The range of markers available to identify HES cells is large and may include determining the presence of any of the following cell markers for a positive identification. Suitable markers for distinguishing ES cells include SSEA-3, SSEA-4, GCTM-2, GDF-3, Cripto (Cr-1 and CR-1) or GDF-3, and genesis. However, none are absolutely specific markers for pluripotent, differentiated or undifferentiated cells. Oct-4 expression is thought to be mainly restricted to pluripotent cells. The use of this marker alone would not be definitive.

Present approaches for the maintenance and proliferation of ES cell populations have limitations. Human ES cells can undergo spontaneous differentiation in vitro necessitating constant removal of such cells for maintenance of pluripotent colonies. Inadequate numbers of ES cells are also a problem for development of their therapeutic potential. These limitations contribute to difficulties in propagating homogeneous HES cells. It is clear that human ES cells are harmed by standard tissue culture propagation techniques that would facilitate their propagation.

Isolation of differentiating cell types from HES cell cultures can ultimately lead to development of cells for transplantation therapies. However, identifying and isolating particular cell populations which are pluripotent or differentiating and destined to particular cell lineages can help in the research and therapeutics of HES cell technology.

Accordingly, it is an object of the present invention to overcome or at least alleviate some of the problems in isolating and identifying HES cell sub-populations and to obtain distinct cell sub-populations from HES cell cultures which can preferably be maintained and sub-cultured in further cell cultures.

### SUMMARY OF THE INVENTION

In a first aspect of the present invention, there is provided a method of identifying a viable sub-population of HES cells, said method comprising:
obtaining a source of HES cells; and
identifying the sub-population of HES cells that are at least GCTM-2 positive.

Applicants have identified the existence of a sub-population of HES cells which are positive for specific HES and preferably express at least one other surface antigen of ES cells including but not limited to TG-30 or CD9. The method described allows for identification of particular cell types within a population of cells having a unique characteristic. These cell types are identified by expression of particular GCTM-2 antigens but lacks expression of other antigens found in a general population of HES cells.

The invention also provides for a sub-population of cells that are substantially homogenous and viable and have the capacity to sub-culture and maintain a high degree of pluripotentiality.

The method is also useful for identifying clusters of cells which are essential for subculturing. These clusters can be identified as being capable of sub-culture and which are viable and have the ability to maintain a high degree of pluripotentiality. Hence, in all cultures where multiple clusters may appear, this method of identification provides for an immediate and convenient detection of cell clumps suitable for propagation to a homogeneous cell population.

In another aspect of thee present invention there is provided a method of isolating a viable sub-population of HES cells, said method comprising:
obtaining a source of HES cells;
identifying those HES cells that are at least GCTM-2 positive; and
selecting for or against those cells which are GCTM-2 positive.

In a preferred aspect of the present invention there is provided a method of isolating a viable sub-population of HES cells, said method comprising:
obtaining a source of HES cells;
exposing the cells to marker of GCTM-2;
reacting the cells to the marker to bind the sub-population of cells to the marker;
separating the sub-population of cells which are bound or unbound to the marker.

The separated cell population has the capacity to continue to grow in culture and maintain a high degree of pluripotenitally.

The marker for GCTM-2 may be an antibody to GCTM-2. Preferably, the marker is a monoclonal antibody to GCTM-2 or a reagent which detects proteoglycans bound to GCTM-2.

In another aspect of the present invention, there is provided an isolated and viable sub-population of HES cells wherein said sub-population is positive for GCTM-2. More preferably, the sub-population is positive for GCTM-2 and Oct-4.

Applicants have found that markers, especially Oct-4, GCTM-2, TRA1-60, TG-30 or CD9 can be used to distinguish sub-populations of HES cells as being differentiated or undifferentiated.

Preferably, the undifferentiated sub-population of cells is distinguished by being positive for GCTM-2. More preferably, the undifferentiated sub-population of cells is positive for Oct-4, and GCTM-2.

In another aspect of the present invention, there is provided a method of subculturing HES cells, said method comprising:
obtaining a source of HES cells;
exposing the cells to a marker of GCTM-2;
reacting the cells to the marker to bind the sub-population or cells to the the marker;
separating sub-populations of HES cells which are bound or unbound to the marker; and
sub-culturing the bound cells.

The invention further provides for a sub-cultured sub-population of HES cells. Preferably the cells are positive for GCTM-2 and more preferably, they are positive for GCTM-2 and Oct-4.

In yet another aspect of the present invention, there is provided a method of transplantation, said method comprising:
obtaining a culture of a substantially pure HES sub-population; and
optionally transplanting the sub-population of cells into a patient; or
optionally, subculturing the sub-population prior to transplanting the cells into a patient.

In another aspect of the present invention there is provided a subpopulation of HES cells which are morphologically distinct from HES cells and having stem cell-like (SCL) characteristics, said cells having the ability to develop into various cell types.

These cells are morphologically distinct from HES cells because they express some but not all of the embryonic stem cell markers characteristic of HES cells.

In another aspect of the present invention there is provided a differentiated epithelial stem cell derived from a HES cell culture.

Preferably, the differentiated epithelial stem cell is an endoderm progenitor capable of differentiating in germ layer cells. More preferably, the cells may differentiate into gut, liver, pancreas or lung.

### FIGURES

FIG. 1 shows phase contrast and epifluorescent micrographs of HES cells. (A). Phase contrast micrograph of a group of HES cells. (B). The same field as A is shown after engaging a filter to show fluorescence associated with Hoechst dye. Hoechst dye stains nuclei of all fixed cells. (C). The same field showing fluorescent staining of the Oct-4 antibody detected by an anti-mouse Ig-FITC secondary antibody. (D). The same field as A showing fluorescent staining of the TRA 1-60 antibody detected by an anti IgM-biotin-streptavidin-Texas Red complex. (E). Phase contrast micrograph of a group of HES cells. (F). The same field as (E) is shown after engaging a filter to show fluorescence associated with Hoechst dye. (G). The same field as (E) showing fluorescent staining of the Oct-4 antibody detected by an anti-mouse Ig-FITC secondary antibody. H. The same field as E showing fluorescent staining of the Tra 1-60 antibody detected by an anti IgM-biotin-streptavidin-Texas Red complex. Scale bar=25 μM.

FIG. 2 shows double immunofluorescent staining of HES cells (FIG. 2A) with Oct-4 antibody detected by an anti-mouse Ig-FITC secondary antibody (FIG. 2B). FIG. 2C shows the same field as A and B but shows fluorescent staining of the TG343 antibody detected by an anti-IgM biotin-streptavidin-Texas Red complex.

FIG. 3 shows HES cells before (A, B) and after (C to F) immunomagnetic separation with GCTM-2 conjugated magnetic beads. (A). Phase contrast micrograph of a group of HES cells taken as start material in the immunomagnetic separation procedure and plated onto mitomycin inactivated MEFS. (B). same field as (A) showing fluorescent staining of the Oct-4 antibody detected by an anti-mouse Ig-FITC secondary antibody. (C). Phase contrast micrograph of the unbound fraction of cells after the immunomagnetic separation procedure. (D). same field as C showing fluorescent staining of the Oct-4 antibody detected by an anti-mouse Ig-FITC secondary antibody in the unbound fraction. (E). Phase contrast micrograph of the bound fraction of cells after the immunomagnetic separation procedure. (F). same field as E showing fluorescent staining of the Oct-4 antibody detected by an anti-mouse Ig-FITC secondary antibody in the bound fraction. Scale bar=25 μM.

FIG. 4 shows HES cells after immunomagnetic separation with GCTM-2 conjugated magnetic beads. (A). Phase contrast micrograph of a group of bound HES cells plated onto mitomycin inactivated MEFS. (B). same field as (A) showing fluorescent staining of the Oct-4 antibody detected by an anti-mouse Ig-FITC secondary antibody. (C). same field as (A) showing fluorescent staining of the Tra 1-60 antibody detected by an anti IgM-biotin-streptavidin-Texas Red complex. (D). Phase contrast micrograph of a group of bound HES cells plated onto mitomycin inactivated MEFS. (E). same field as (D) showing fluorescent staining of the Oct-4 antibody detected by an anti-mouse Ig-FITC secondary antibody. (F). same field as (D) showing fluorescent staining of the TRA 1-60 antibody detected by an anti IgM-biotin-streptavidin-Texas Red complex. Scale bar=25 μM.

FIG. 5A shows that GCTM-2 isolated cells maintain Oct-4 expression characteristics after 24 hours and 120 hours in culture.

FIG. 5B shows that GCTM-2 isolated cells maintain GCTM-2 characteristics after 120 hours in culture.

FIG. 6 shows RT-PCR analysis of gene expression in bound HES cells after immunomagnetic separation with GCTM-2 conjugated magnetic beads. RT-PCR products were run on 1.5% agarose gels and stained with ethidium bromide. Lane 1, 100 bp DNA ladder. Lane 2 PCR for β-actin carried out with omission of reverse transcriptase. Lane 3, RT-PCR for β-actin (200 bp). Lane 4, RT-PCR for Oct-4 (320 bp).

FIG. 7 shows stem cell like (SCL) cells under phase contrast microscopy. The SCL cells have a distinct morphological appearance and could be identified with a translucent cytoplasm and darkened nucleus (FIG. 7A). After a further seven to ten days in culture other cell types appear within the colony as cells begin to differentiate (FIG. 7B).

FIG. 8 shows indirect immunofluorescence microscopy of the SCL cells showing negative staining for GCTM2 (FIG. 8A), TRA-160 (FIGS. 8B-D), TG343 (FIG. 8E). FIG. 8F shows double label staining of a colony of SCL cells for GCTM-2 (red) and Oct-4 (green). Cells at the rim of the colony express both markers, but most cells within the colony express Oct-4 but not GCTM-2.

FIG. 9 shows cells displaying positive fluorescent staining for TG30 (FIG. 9A) and antibody CAM 5.2 against low molecular weight cytokeratins (FIG. 9B) as well as a low intensity fluorescence for the intermediate filament marker Vimentin (FIG. 9C) and the cell surface marker CD9 (9D). No positive fluorescence was observed for SSEA-1, PHM4 (HLA antigen) and the epithelial marker EpCAM (clone Ber-EP4; DAKO).

FIG. 10 shows expression of genes known to be transcriptionally active within pluripotent stem cell populations including Oct-4, Cripto (FIG. 10A) and Genesis (FIG. 10C). Transcripts for the neural marker Pax 6 (FIG. 10B) and relatively low intensity transcripts for hepatocyte nuclear factor (HNF)-3α (FIG. 10C) were also detected, as they are in conventional ES cell cultures. There were no transcripts for the endoderm genes alphafetoprotein, vitronectin, or transferrin, nor for the neural marker nestin, although verification of successful RT-PCR was validated by identification of the housekeeping gene actin.

FIG. 11 shows SCL cells (FIG. 11A) are capable of differentiating into neurospheres (FIG. 11B) and epithelial-like cells (FIG. 11C).

FIG. 12 shows cells after two or three days with rounded cell colonies.

FIG. 13(A, B) shows the putative endodermal progenitor at seven to ten days.

FIG. 14 shows induction of the endodermal progenitors cells by cultivation of human ES cells in low glucose medium in the presence of nicotinamide. FIG. 14A shows control cells grown in low glucose medium without nicotinamide; flattened squamous cells commonly seen under suboptimal culture conditions predominate. 14B shows cells grown in low glucose medium plus nicotinamide; endodermal progenitors predominate.

FIG. 15 shows immunostaining of the progenitor cells and differentiated cells derived from them. A, Ep-CAM; B, cytokeratin 8; C, A33 antigen; D, alphafetoprotein; E, albumin; F, GCTM-5 antigen.

FIG. 16 shows RT-PCR analysis of endodermal markers in the novel endodermal progenitor cells.

### DESCRIPTION OF THE INVENTION

In a first aspect of the present invention, there is provided a method of identifying a viable sub-population of HES cells, said method comprising:
obtaining a source of HES cells; and
identifying the sub-population of HES cells that are at least GCTM-2 positive.

Applicants have identified the existence of a sub-population of HES cells which are positive for specific HES markers and preferably express at least one other surface antigen of ES cells including but not limited to TG-30 or CD9. The method described allows for identification of particular cell types within a population of cells having a unique characteristic. These cell types are identified by expression of particular GCTM-2 antigens but lacks expression of other antigens found in a general population of HES cells.

The present method of identification of a sub-population of HES cells provides a rapid and reproducible method for identifying such cell populations. Different subpopulations exist in a HES cell source and they may exist as differentiated and undifferentiated cells. However, they can be identified by the presence of markers which are expressed on the cell at various stages of development. Whilst several markers have been used to identify HES cells in a general population, applicants have found that various sub-populations of HES cells can be identified by specific markers, in the absence of other HES cell markers which would generally be present. This possibly indicates that HES cells in culture can progress on mass or in sub-populations by expressing predominantly one marker which is indicative of a period of development or is stage specific. Sub-populations may be identified on this basis. Various markers will be indicative of an undifferentiated or differentiated state. Spontaneous differentiation often occurs in HES cultures resulting in non-homogeneous cell cultures. Hence it is desirable to obtain cell cultures of greater homogeneity to improve or to aid in research in HES technology. Preferably this method identifies undifferentiated HES cells.

The HES cells as used in the present invention may derive from any source. They may be a crude fraction derived directly from embryos or preferably, the HES cells are derived from a culture of HES cells. Preferably the culture of HES cells is prepared by methods outlined in PCT/AU99/00990 or PCT/AU01/00278 by the applicants, the contents of which are incorporated herein by reference. Various sources of HES cells are also discussed in these applications and are incorporated herein.

Preferably, the HES cells are dissociated to yield a suspension of cells. Most preferably, the cells are dissociated to yield a suspension of clumps of cells comprising approximately 10 to 100 cells per clump. Any method of dissociation may be utilised including mechanical and chemical dissociation. Preferably, the cells are dissociated using an enzyme such as dispase followed by dispersion of the cells by trituration.

Throughout the description and claims of this specification, the word “comprise” and variations of the word, such as “comprising” and “comprises”, is not intended to exclude other additives, components, integers or steps.

HES specific markers are generally used to identify populations of HES cells. However, the present invention utilises markers specific for cell types. GCTM-2 has been identified by the applicants to be present on HES cells which are viable and capable of further propagation. More preferably, the GCTM-2 is expressed in the absence of other HES cell markers. For instance, Oct-4 is a marker which indicates a degree of differentiation and is down regulated upon differentiation. Applicants have found that in a population of Oct-4⁺ cells, not all express GCTM-2. Hence applicants have found a means to identify a sub-population of cells based on expression of GCTM-2.

The canonical definition of a primate embryonic stem cell has included the surface expression of the proteoglycan carrying the TRA1-60 and GCTM-2 and expression of the SSEA3 and 4 epitopes. Stage-specific embryonic antigens 1, 3 and 4 are globoseries glycolipids recognised by monoclonal antibodies originally raised to identify particular stages of mouse embryo development. SSEA-3 and SSEA-4 but not SSEA-1 are expressed by non-human primate ES cells and human ES and EC cells. This expression disappears upon differentiation and SSEA-1 expression increases. Several epitopes identified by a group of antibodies reactive with a pericellular matrix proteoglycan characteristically present on the cell surface of human EC cells are also expressed by human and non-human primate ES cells. TRA 1-60 is a sialidase-sensitive epitope on this molecule and GCTM-2 and TG343 react with epitopes on its core protein. Antibody TG30 recognises a 25 kDa protein which copurifies with the keratan sulphate proteoglycan and binds to the surface of primate pluripotent stem cells. However within the stem cell population, not all cells express all of these markers. Thus, the true pluripotential ES cell may only be identified through the isolation of cells with specific markers on their surface and the subsequent demonstration that the isolated cells are indeed pluripotent. It is possible that cells expressing different combinations of surface markers represent various stages of stem cell differentiation. Definition of these subpopulations, and their isolation from mixed cell cultures, may be advantageous. Separation of pluripotent cells from their differentiated progeny, either by positive or negative selection, may eliminate inhibitory influences of the differentiated cells on the stem cells. Or, isolation of cells with more restricted differentiation potential may facilitate selection and growth of desired types of progenitor cell.

Preferably, the GCTM-2 marker is identified along with the Oct-4 marker. More preferably, the GCTM-2 marker is identified along with the Oct-4 and/or TRA1-60 marker, TG-30 or CD9 marker.

A characteristic property of embryonic stem cells is expression of the POU domain transcription factor Oct-4. Oct-4 is expressed in the ICM of mouse blastocysts and down-regulated as epiblast cells lose pluripotentiality. As predicted from the expression and function of Oct-4 in the mouse embryo, Oct-4 is transcriptionally active in human ES cells, becoming inactive within differentiated cell lineages. As such Oct-4 expression is a consistent marker in the identification of pluripotent cell types but not for isolation. A member of the EGF-CFC family, Cripto (Cr-1) is detectable in the mouse ICM of the blastocyst at day 4 of development and in undifferentiated F9 embryonal carcinoma cells (Saloman et al., 2000). Human Cripto (CR-1) or teratocarcinoma-derived growth factor-1 (TDGF-1) is also expressed by an undifferentiated EC cell line, NTERA2/D1. Expression of both CR-1 and Cr-1 is lost in NTERA2/D1 and F9 cells following retinoic-acid induced differentiation. A transcriptional repressor Genesis, a member of the Winged Helix/Forkhead family of transcription factors has been shown to have expression restricted to mouse ES cells and human EC cells that is down-regulated upon differentiation (Sutton et al., 1996).

Preferably, the method identifies a viable sub-population of undifferentiated or pluripotent HES cells. The undifferentiated cells maintain a high degree of pluripotentiality and preferably have the capacity to propagate in multiple cultures. They preferably can be passaged to maintain the cell line in a substantially homogeneous culture.

The method is also useful for identifying clusters of cells which are essential for sub-culturing. These clusters can be identified as being capable of sub-culture and which are viable and have the ability to maintain a high degree of pluripotentiality. Hence, in all cultures where multiple clusters may appear, this method of identification provides for an immediate and convenient detection of cell clumps suitable for propagation to a homogeneous cell population.

Methods of identification may be by any methods available to the skilled person. Where the methods are directed to identifying the antibody GCTM-2, Oct-4 and/or TRA1-60, the methods of identification are directed to at least detecting the GCTM-2 antigen. An effective means of identifying this antigen is by use of GCTM-2 antibodies, preferably monoclonal antibodies to GCTM-2 are used. Preferably, the means to identify GCTM-2 antigen is a means which does not harm the cell and allows the cell to be subcultured and propagated. Cell surface markers are preferred as this causes less harm to the cells and allows for continued propagation of the cells.

The methods of detection may also include the addition of a label or other detector molecule on the antibody to facilitate the identification of cells expressing GCTM-2.

In another aspect of the present invention there is provided a method of isolating a viable sub-population of HES cells, said method comprising:
obtaining a source of HES cells;
identifying those HES cells that are at least GCTM-2 positive; and
selecting for or against those cells which are GCTM-2 positive.

In a preferred aspect of the present invention there is provided a method of isolating a viable sub-population of HES cells, said method comprising:
obtaining a source of HES cells;
exposing the cells to a marker of GCTM-2;
reacting the cells to the marker to bind the sub-population of cells to the marker;
separating the sub-population of cells which are bound or unbound to the marker.

Based on the methods for identifying sub-populations of HES cells which identify those cells which are GCTM-2 positive and to which may lack other HES cell markers, the method of isolation can utilise these identification methods to isolate the same sub-population of cells.

The method of isolating as described herein isolates a sub-population of HES cells. Depending on the markers used, various sub-populations may be isolated. The applicants have identified the existence of a sub-population of HES cells which are GCTM-2 positive and may lack other HES cell markers. Preferably the cells have the ability to be propagated and may be passaged in multiple cultures and generations. Preferably, the methods isolate differentiated and undifferentiated cells. More preferably, the method isolates undifferentiated cells or pluripotent cells, which upon propagation and passage can maintain pluripotentiality.

Any method of identifying cells that are at least GCTM-2 positive may be employed and may be as described above. Preferably, antibodies to the GCTM-2 are employed to identify those cells expressing this antigen. Alternatively, methods that can identify the proteoglycan that binds to GCTM-2 may be used to identify cells that express GCTM-2.

Selection of the cells that are GCTM-2 positive maybe dependent upon the method of identification.

In another preferred aspect of the present invention, there is provided a method of isolation of viable sub-populations of HES cells, said method comprising:
obtaining a source of HES cells;
exposing the cells to a support, wherein said support is coupled to an antibody for GCTM-2;
reacting the cells to the antibody to bind the sub-population of cells to the support via the antibody; and
separating bound and unbound sub-populations of HES cells.

Where the method of identification relies on an antibody to GCTM-2, the antibody may be coupled to a static or mobile support. Panning cells on a culture plate or using particles or beads which have been treated with the antibody may be used.

Preferably, the HES cells are exposed to a magnetic particle. The magnetic particle or support may be any particle to which a marker of GCTM-2 can be attached or coupled. Preferably, the magnetic particles are magnetic beads. The beads may be obtained from any source providing they can be modified to attach the marker. A suitable source of beads is from Dynal, Oslo, Norway. Any solid substrate with an antibody attached to it may be used to isolate clusters of ES cells bearing the antibody,

The particles, preferably beads, may be modified in any way which facilitates attachment of the marker for GCTM-2. Preferably, the bead is modified with an antibody to GCTM-2. However, in some cases, a marker may be a protein or compound which reacts to a receptor on the HES cell or to the GCTM-2 antigen. In these cases, the magnetic beads may be modified with a complimentary attachment to facilitate attachment of the HES cell population to the particle via the marker.

Most preferably, the particle is modified by coating with an anti-IgM which is particularly suitable for use with the stem cell reactive monoclonal antibody GCTM-2.

Preferably, the magnetic particles are pre-prepared magnetic beads with anti-IgM. The marker, generally a monoclonal antibody can then be reacted with the beads by methods familiar to the skilled addressee to provide immunomagnetic beads useful for selecting for or against sub-populations of HES cells.

The marker of the HES cell sub-population may determine how the magnetic particle is modified to accept the marker or the type of magnetic particle used. For instance, if the marker is a monoclonal antibody, a suitable particle will have an anti-immunoglobulin to facilitate coupling of the monoclonal antibody marker to the particle. Markers of HES cells may be selected from the group including, including but not limited to, SSEA-3, SSEA-4, GCTM-2, TRA1-60, TG 30, TG343 or Cripto. However, applicants have found that to isolate the viable sub populations of HES cells, the markers should at least be for GCTM-2. Other HES cell markers may be used in conjunction with GCTM-2. Other suitable markers include those for Oct-4 and TRA 1-60, TG-30 or CD9.

In a preferred embodiment of this aspect of the invention, there is provided a method of isolation of a viable sub-population of HES cells said method comprising:
obtaining a source of HES cells which are dissociated to yield a suspension of cells in clumps of approximately 10 to 100 cells;
exposing the cells to a marker for GCTM-2;
reacting the cells to the marker to bind the sub-population of cells to the marker; and
separating bound and unbound sub-populations of HES cells.

The marker will depend on the sub-population to be targeted and isolated.

More preferably, the marker is GCTM-2 and Oct-4 and/or TRA1-60, TG-30 or CD9. It is preferable that the method isolates undifferentiated cells which are bound to the particles. However, the population of cells which are not bound may also provide another sub-population which may comprise predominantly differentiated cells. Accordingly, the essence of the invention provides for a separation of sub-populations of HES cells which are preferably differentiated or undifferentiated. Most preferably they are undifferentiated cells.

Alternatively, in a preferred aspect the present method may be used to isolate sub-populations of differentiated HES cells using markers indicative of differentiated cells. For instance, SSEA-3 and SSEA-4 but not SSEA-1 are expressed in HES cells. This expression disappears upon differentiation and SSEA-1 expression increases. This marker may therefore be bound to the anti-IgM magnetic particle for isolation of the undifferentiated cells away from differentiated cells.

These selection techniques may be used positively or negatively wherein one sub-population is isolated via attachment to the magnetic particle whilst the other sub-population remains unbound. Both populations (bound and unbound) may be further sub-cultured or used for research purposes.

Once the cells are reacted to the marker to bind with the particle, they may be separated using any particle concentrator. Magnetic particles may be separated using the Dynal Magnetic Particle Separator (MPC). However, any magnetic separation that can, separate the magnetic particles from a cell suspension may be used.

Any method of cell separation may be further employed to separate pure or single cells. Flow cytometry may be employed.

In another aspect of the present invention, there is provided an isolated and viable sub-population of HES cells wherein said sub-population is positive for GCTM-2. More preferably, the sub-population is positive for GCTM-2 and Oct-4 and/or TRA 1-60, TG-30 or CD9.

In another aspect of the present invention there is provided an isolated and viable sub-population of HES cells said subpopulation being negative for GCTM-2. More preferably, the sub-population is negative for GCTM-2 and Oct-4 and/or TRA 1-60, TG-30 or CD9.

Applicants have found that markers, especially Oct-4, GCTM-2 and TRA1-60, TG-30 or CD9 can be used to distinguish sub-populations of HES cells as being differentiated or undifferentiated.

Preferably the sub-population comprises substantially pure differentiated or undifferentiated HES cells. Most preferably, the cells are undifferentiated and retain pluripotential activity.

Preferably, the undifferentiated sub-population of cells is distinguished by being positive for GCTM-2. More preferably, the undifferentiated sub-population of cells is positive for Oct-4 and GCTM-2.

In a preferred aspect, the isolated and viable sub-population of HES cells is prepared by the methods described herein.

In a further aspect of the present invention there is provided an isolated and viable sub-population of HES cells capable of sub-culture said sub-population comprising HES cells that are positive for GCTM-2. More preferably, the sub-population is positive for GCTM-2 and Oct-4 and/or TRA 1-60, TG-30 or CD9.

Applicants have found that cells expressing such markers have a propensity to be capable of being serially sub-cultured. Preferably, the sub-culture is of undifferentiated cells.

In another aspect of the present invention there is provided an isolated and viable sub-population of HES cells capable of sub-culture said sub-population comprising HES cells that are negative for GCTM-2. More preferably, the sub-population is negative for GCTM-2 and Oct-4 and/or TRA 1-60, TG-30 or CD9.

These cells may also be sub-cultured. However, they may have less homogeneity but nevertheless, they are capable of serial subculture. These sub-populations will preferably have cells predominantly in the differentiated state. They may be capable of further developing into somatic cells of various types.

In another aspect of the present invention, there is provided a method of subculturing HES cells, said method comprising:
obtaining a source of HES cells;
exposing the cells to a marker of GCTM-2;
reacting the cells to the marker to bind the sub-population of cells to the the marker;
separating sub-populations of HES cells which are bound or unbound to the marker; and
subculturing the bound cells.

Applicants have found that certain sub-populations in a HES source can be sub-cultured successfully. These cells can be isolated by methods which identify various markers on the cell surface generally indicating a cell type in a particular phase of development. By extracting these particular cells, HES cells can be serially sub-cultured.

In another preferred aspect of the present invention, there is provided a method of subculturing HES cells, said method comprising:
obtaining a source of HES cells;
exposing the cells to a support coupled to at least one marker for GCTM-2;
reacting the cells to the marker to bind the sub-population of cells to the marker;
separating bound and unbound sub-populations of HES cells; and
sub-culturing the bound cells.

Applicants have found that cells which are capable of further sub-culture are identified by the marker GCTM-2, and preferably Oct-4, GCTM-2 and TRA1-60. The immunoisolation procedure enriches for clusters of cells which are positive for Oct-4 and these cells have been found to be viable and can reattach to a monolayer (feeder layer) after overnight culture. Clustered cells have been found to be positive for Oct-4 and for GCTM-2 and TRA1-60 thereby indicating a propensity for these cells to grow in coherent clusters. These cells have been found to maintain stem cell properties upon serial cultivation. Preferably the cells which can be isolated and further sub-cultured are GCTM-2⁺, preferably the cells are both GCTM-2⁺ and Oct-4⁺.

This procedure allows for cultivation of defined populations without contamination of minority populations of differentiated cells. Preferably the sub-population that is subcultured is an undifferentiated sub-population or HES cells.

Hence a sub-population is firstly isolated and then sub-cultured. The identified and isolated cell population has the propensity to be sub-cultured. The cells are identified and isolated via a marker of HES which is a marker for GCTM-2.

The marker is preferably selected from the group including Oct-4, GCTM-2 or TRA1-60. Most preferably, the marker is GCTM-2, or TRA 1-60, most preferably the marker is GCTM-2.

In a preferred aspect, and for the process of sub-culturing, a HES cell population which is a cell suspension of clusters of ES cells, is subjected to magnetic particles coated with the appropriate markers for the sub-population. However, any solid support may be used. The cells may be suspended in a medium which facilitates binding of the sub-population of HES cells to the marker coupled to the magnetic particles.

Once the cells and particles have bound, separation techniques familiar to the skilled addressee using particle concentrators such as the Dynal MPC may be used to concentrate the particles away from unbound cells and supernatant. This latter subpopulation of cells may form the basis of another sub-culture.

Bound cells may be plated directly onto tissue culture plates or some cells saved for RT-PCT analysis or further identification and characterization. Cells may be incubated at 37° C., 5% CO₂ in a humidified chamber.

In another aspect of the present invention, there is provided a sub-cultured sub-population of HES cells. Preferably, the sub-population comprises HES cells being positive for GCTM-2. More preferably, the sub-population is positive for GCTM-2 and Oct-4.

In another preferred aspect of the present invention there is provided a sub-cultured sub-population of HES cells wherein the sub-population comprises HES cells being negative for GCTM-2. More preferably, the sub-population is negative for GCTM-2 and Oct-4.

Preferably, the subcultured sub-population is a substantially pure population of viable differentiated or undifferentiated cells. More preferably, the sub-population comprises substantially pure undifferentiated HES cells that maintain a pluripotential character. Preferably the undifferentiated cells are identified as being GCTM-2⁺ and preferably GCTM-2⁺ and Oct-4⁺.

Preferably, the sub-population of HES cells is prepared by sub-culturing the sub-populations of cells described herein. More preferably, the sub-cultured sub-population is prepared by the methods described herein.

The sub-populations of HES cells may be used to scale-up for therapeutic or research purposes. Once the cells are isolated and have the propensity for sub-culture, they may be expanded using techniques available to the skilled addressee.

In yet another aspect of the present invention, there is provided a method of transplantation, said method comprising:
obtaining a culture of a-substantially pure HES sub-population; and
optionally transplanting the sub-population of cells into a patient; or
optionally, subculturing the sub-population prior to transplanting the cells into a patient.

Because of the methods outlined herein, scale-up of sub-populations of cultures to large cultures can provide sufficient cells for transplant purposes and to develop therapeutic or research potential.

Preferably the HES sub-populations are differentiated or undifferentiated HES cells. More preferably the HES sub-populations are prepared by the methods described herein. Where the cells are undifferentiated cells these may be induced to differentiate under normal differentiation conditions. They may be induced to differentiate in vivo or in vitro. Where the cells are induced to differentiate in vitro they may do so by methods described in PCT/AU01/00278 or PCT/AU01/00735 by the applicants, of which the entire contents are incorporated herein by reference.

However, other differentiation signals may be employed and are available to the skilled addressee. Differentiation may also be induced by cultivating to a high density in monolayer or on semi-permeable membranes so as to create structures mimicing the postimplantation phase of human development, or any modification of this approach. Cultivation in the presence of cell types representative of those known to modulate growth and differentiation in the vertebrate embryo (eg. endoderm cells or cells derived from normal embryonic or neoplastic tissue) or in adult tissues (eg. bone marrow stromal preparation) may also induce differentiation, modulate differentiation or induce maturation of cells within specific cell lineage so as to favour the establishment of particular cell lineages.

Chemical differentiation may also be used to induce differentiation. Propagation in the presence of soluble or membrane bound factors known to modulate differentiation of vertebrate embryonic cells, such as bone morphogenetic protein-2 or antagonists of such factors, may be used.

The cells may also be genetically modified prior to transplantation and/or differentiation and then transplantation.

The differentiated or undifferentiated cells may be used as a source for isolation or identification of novel gene products including but not limited to growth factors, differentiation factors or factors controlling tissue regeneration, or they may be used for the generation of antibodies against novel epitopes. The cell lines may also be used for the development of means to diagnose, prevent or treat congenital diseases.

Much attention recently has been devoted to the potential applications of stem cells in biology and medicine. The properties of pluripotentiality and immortality are unique to ES cells and enable investigators to approach many issues in human biology and medicine for the first time. ES cells potentially can address the shortage of donor tissue for use in transplantation procedures, particularly where no alternative culture system can support growth of the required committed stem cell. ES cells have many other far reaching applications in human medicine, in areas such as embryological research, functional genomics, identification of novel growth factors, and drug discovery, and toxicology.

In yet another aspect of the present invention there is provided a method of identifying gene expression in a HES cell, said method comprising:
obtaining a substantially homogenous HES cell population; and
conducting gene expression analysis on the population.

Once a substantially homogeneous population of cells is obtained they may be used to analyse gene expression in the isolated cell populations, by any one of a number of techniques known to the skilled addressee.

A problem with gene analysis is the availability of a homogenous source of DNA from a homogenous source of cells. For analysis of HES populations, current cultures contain heterogenous populations of HES cells which are at varying degrees of differentiation. Hence the nucleic acid is not obtained from a homologous population. The present invention recognises that sub-populations of HES cells can be identified based on cell markers, particularly, GCTM-2, Oct-4 and TRA1-60, TG-30 or CD9.

Preferably, the HES cell population is obtained by methods described herein. This provides HES cells of differentiated or undifferentiated status which provides for analysis of gene expression, preferably during the process of differentiation and/or proliferation.

The present method provides for an enrichment process which can enrich for cells of a particular characteristic sub-population from a heterogeneous HES cell population. The advantage is that all cells are substantially the same with a similar degree of differentiation. Cells can be induced to differentiate by methods familiar to the skilled addressee and gene analysis conducted on the differentiating cells.

The present invention may be used to improve the growth and yield of stem cells from human ES cell cultures. Without being limited by theory, it is postulated that by separating stem cells from differentiating cells, the invention removes a source of inhibitory signals which may cause stem cells to differentiate or die. It is known that differentiating cells in ES cell cultures produce these signals.

Gene expression can be analysed by gene arrays of which the methods are familiar to the skilled addressee. Any standard arrays may be used. However, the ability to use a repeatable cell population and DNA source provides a more reproducible analysis of gene expression in HES cell populations.

In another aspect of the present invention there is provided a subpopulation of HES cells which are morphologically distinct from HES cells and having stem cell-like (SCL) characteristics, said cells having the ability to develop into various cell types.

The term stem cell-like (SCL) as used herein means that the cell has many of the properties of stem cells which distinguish stem cells from other cells. These properties include pluripotentiality and the ability to differentiate into all cells of the body.

These cells are morphologically distinct from HES cells and they express some but not all of the embryonic stem cell markers characteristic of HES cells. The cells show negative staining by indirect immunofluorescence microscopy for GCTM-2, TRA1-60, TG343 and SSEA-4. The cells show positive staining for TG30 and antibody CAM5.2 against low molecular weight cytokeratins as well as low intensity fluorescence for the intermediate filament marker vimentin. They express the tetraspannin cell surface marker CD9, required in the mouse for maintenance of ES cell pluripotentiality. Preferably, no positive fluorescence is evident in these cells for SSEA-1, PHM4 (HLA antigen) and the epithelial marker EpCAM.

These cells may be isolated by immunological methods employing techniques described above. Thus cells which are TG30 positive, GCTM-2 negative may be isolated using a combination of negative and positive immunoselection from a culture of ES cells grown under standard conditions.

Preferably the cells can be further identified by expression of genes known to be transcriptionally active within pluripotent stem cell populations including Oct-4, Cripto, Pax6 and Genesis. Preferably, no transcripts for the endoderm genes alphafetoprotein, vitronectin, or transferrin, nor for the neural marker nestin, are present in these cells.

The SCL cell as described herein is a novel type of pluripotent cell which derives from, or is a precursor of, the canonical ES cell. The SCL cell may have desirable properties compared to conventional ES cells, such as ease of growth during routine subculture or cloning, or more extensive capacity for in vitro differentiation into diverse lineages, or greater susceptibility to uptake and integration of DNA constructs.

The SCL cell also has the ability to differentiate in vitro to form neurospheres, neural cells, endothelial cells or extraembryonic endoderm by spontaneous differentiation. Neurospheres may form spontaneously in the presence of neurobasal media as described in PCT/AU01/00278.

This novel SCL cell population has advantageous proliferative properties and thus, may be advantageous in its growth characteristics and thus, have a practical use for future development of therapeutic applications for human ES cell therapies.

In another aspect of the present invention there is provided a method of isolating a stem cell-like (SCL) cell which is morphologically distinct from HES cells and having stem cell-like (SCL) characteristics, said cells having the ability to develop into various cell types said method comprising:
obtaining a differentiating HES cell population comprising spontaneously differentiated HES cells;
identifying SCL cells by negative expression of cell surface markers characteristic of SCL cells including GCTM-2, TRA 1-60, TG343 and SSEA-4 and positive expression of TG 30 antigen; and
isolating said SCL cells expressing said negative expression.

Preferably, the HES cell population of differentiated HES cells may be prepared by the methods described herein or may be obtained from a crude fraction derived from embryos or blastocysts.

Without being limited by this example of a suitable procedure for the isolation, the SCL cells may be isolated by the following procedure. SCL cells are isolated from differentiating human embryonic stem (ES) cell colonies six weeks following plating and may be transferred onto a Mitomycin C treated embryonic fibroblast cell line, preferably a mouse embryonic fibroblast cell line, more preferably a mouse STO cell cell line. Preferably, the fibroblast cell line is cultured in DMEM medium (Gibco, without sodium pyruvate, with glucose 4500 mg/L) supplemented with 10% (v/v) Fetal Bovine Serum (FBS; CSL, Utah), β-mercaptoethanol (0.1 mM; GIBCO), 1% (v/v) Non Essential Amino Acids (NEAA; GIBCO), 50 ng/ml basic FGF, 1% (v/v) insulin, selenium and transferrin solution (ITS, GIBCO), glutamine (2 mM; GIBCO), penicillin and steptomyocin (50 μg/ml; GIBCO).

From approximately seven days after plating, the stem cell colonies may be further propagated by isolation of the cells from the surrounding areas of differentiating cells. These cells may be identified by the characteristics described above in particular with respect to the immunofluorescent staining pattern and gene expression. The cells are also spontaneously differentiated cells from the HES population. Mechanical isolation is preferred. The culture medium may be aspirated leaving the cells to be washed preferably with phosphate-buffered saline containing calcium and magnesium. The SCL stem cell colonies may be extracted and treated with dispase at approximately 10 mg/ml in culture medium. These cells may then be replated on a new fibroblast layer.

To induce further differentiation into neurospheres, the cells may be cultured under conditions described in PCT/AU01/00278.

In another aspect of the present invention there is provided a differentiated epithelial stem cell derived from a HES cell culture.

The ability of human ES cells to develop into a diverse array of adult cells was clearly demonstrated by the presence of tissue representative of all three germ layers within tumours formed after ES cell transfer into the testis capsule of severe immunodeficient (SCID) mice (Reubinoff et al., 2000). Differentiated cell types seen included cartilage, squamous and glandular epithelium, muscle and bone. Specific cells within differentiating ES cells cultures, can be isolated, and, when exposed to conditions which support the growth of neural stem cells can give rise to a progenitor which can differentiate into mature neurons. Thus, factors identified as critical to the development of the early mammalian embryo can be employed to direct human ES cell differentiation, supporting the hypothesis that spontaneous ES cell differentiation is a caricature of normal embryonic development.

In the mammalian embryo, the embryonic endoderm emerges during gastrulation to form the germ layer which will give rise to the primitive gut. This tissue then undergoes specialisation along its length to form the precursors of the gut, the liver, the pancreas the lungs and other tissues. There is evidence in the mouse that multipotent endodermal cells may be isolated from the embryo, but these have not been serially cultivated in the laboratory (Gualdi et al., 1996). Human ES cells can develop into embryonic endodermal structures such as gut-like tissue and respiratory epithelium within teratomas following transplantation into SCID mice. Human ES cells differentiate into endodermal cells by detection of alphafetoprotein (AFP) assayed in supernatant.

The present invention provides a novel cell type from differentiating human ES cells, that has both morphological and molecular epithelial characteristics and that expresses some but not all early endoderm markers. Isolation and characterisation of a novel cell from differentiating human ES cells could lead to development of cells for transplantation therapies. These cells may be distinguished by a distinctive morphological appearance with darkened cytoplasm and translucent nucleus. They are identified in culture by thin, fibroblastic cells surrounding the epithelial-like cell colonies. Some colonies may be associated with a more rounded cell that detaches from the epithelial-like cells as proliferation continues. Preferably, cell growth is enhanced by addition of cholera toxin to the culture medium.

Preferably, the differentiated epithelial stem cell is an endoderm progenitor capable of differentiating in germ layer cells. More preferably, the cells may differentiate into gut, liver, pancreas or lung.

The endodermal progenitor cells may be isolated from spontaneously differentiating human ES cell cultures from 4-8 weeks after subculture. Alternately, the human ES cells may be induced to differentiate into the endodermal progenitors by altering the growth medium. One week to 10 days after subculture of the ES cells under standard conditions, the growth medium is switched to one which contains a low glucose formulation of DMEM with sodium pyruvate, plus 10 mM nicotinamide, and 5-10% foetal calf serum. Growth of the ES cells in this medium will convert at least 50% of the cultures to endodermal progenitors within two weeks.

The cells may be identified by indirect immunofluorescence methodology. Preferably, the cells show positive staining for the epithelial cell markers EpCam and/or low molecular weight cytokeratins, albumin and GCTM-5. Cells stain negatively for GCTM-2. Preferably, a further feature includes observing isolated patches of cells above the plane of the monolayer which show expression of the endoderm marker alphafetoprotein and/or the gut cell specific A33 antibody. More preferably, no significant staining is observed for the markers selected from the group including PHM4 (HLA Class I antigen), CD34 or the stem cell marker Oct-4. However, the cells are positive for genesis, Hex, Sox-17, α-1 anti-trypsin.

The cells may be further distinguished by the expression of hepatocyte nuclear factor (HNF)-3α, and HNF-4. Preferably there is no evidence of gene expression for Oct-4 or CR-1, characteristic of pluripotent stem cell populations. Transcripts for Pax 6 may also observed. Pax 6 is primarily expressed in neural tissues during embryo development.

Thus the novel cells are clearly epithelial in nature. They are no longer pluripotent stem cells; they lack all markers of pluripotent human stem cells and fail to show the typical multilineage differentiation shown by ES cells in vitro. The response of this cell to cholera toxin and its requirement for STO cells further distinguish it from human ES cells which are not affected by cholera toxin and which do not grow well on STO monolayers.

In another aspect of the present invention there is provided a somatic cell differentiated from the epithelial stem cell as herein described.

The expression in some colonies of the markers AFP (made by embryonic endodermal tissue) and the A33 antigen, the embryonic hepatocyte marker defined by monoclonal antibody GCTM-5, as well as expression of HNF3 alpha and HNF-4, suggest that these primitive epithelial cells may be capable of differentiation into several embryonic endodermal derivatives. The cells may respond to various known inducers of endodermal differentiation by forming different derivatives, preferably selected from the group including gut, liver, lung and pancreas. They may provide an in vitro source of these important cell types.

In another aspect of the present invention there is provided a method of isolating an epithelial stem cell, said cells having the ability to develop into various cell types said method comprising:
obtaining a differentiating HES cell population comprising differentiating HES cells;
identifying epithelial stem cells morphologically and by expression of cell surface markers; and
isolating said cells and culturing the cells on a STO mouse fibroblast layer in a HES media including cholera toxin.

Preferably, the HES cell population of differentiated HES cells is prepared by the methods described herein. However, the HES differentiating cells may also be obtained directly from an embryo or blastocyst or crude preparations of the embryo or blastocyst.

The cells are isolated from HES colonies comprising differentiated HES cells and SCL cell colonies as herein described.

Without being limited by this example of a suitable procedure, the differentiated epithelial stem cell may be isolated by the following procedure. Epithelial-like cells, identified morphologically, may be isolated from crude HES cell populations, cultures of HES cell populations, differentiating HES cell colonies and stem-cell like (SCL) colonies six to eight weeks following subculture. Sub-populations of differentiating cells may be prepared as described above. Cells may be identified by the characteristics described above and then may be transferred onto a Mitomycin C treated embryonic fibroblast cell line, preferably a STO fibroblast cells line and cultured in DMEM medium (Gibco, without sodium pyruvate, with glucose 4500 mg/L) supplemented with 20% (v/v) Fetal Bovine Serum (FBS; Hyclone, Utah), β-mercaptoethanol (0.1 mM; GIBCO), 1% (v/v) Non Essential Amino Acids (NEM; GIBCO), 1% (v/v) insulin, selenium and transferrin solution (ITS, GIBCO), glutamine (2 mM; GIBCO), penicillin and steptomyocin (50 μg/ml; GIBCO). Preferably, the culture medium is supplemented with Cholera Toxin (0.02 μg/μl) or Stem Cell Factor (SCF, 25 ng/μl). Six to eight weeks after plating the epithelial-like cell colonies may be further propagated by dispersing the cells into smaller clumps. This may be achieved by any cell dispersion method.

Approximately twenty-four hours following transfer, clusters of small flat cells may appear outgrowing from cell clumps. Within approximately two to three days rounded cell colonies may form providing the epithelial cell line. These may be identified morphologically and be distinguished by the features described above including the expression of cell surface markers and RT-PCR.

The cells are preferably grown on Matrigel™ or collagen coated dishes as this helps to maintain their specific phenotype.

The epithelial-like cells can further differentiate into liver cells as evidenced by expression of liver markers such as α-1 anti-trypsin, albumin, c-met and GCTM-5. Expression of this latter marker is consistent with embryonic liver. Accordingly, the present invention provides a liver cell differentiated from the epithelial cell line. These cells may be isolated from endodermal progenitor cultures by immunological techniques described above.

These hepatocyte cells may undergo differentiation into other endodermal cells types including pancreatic cells.

The present invention will now be more fully described with reference to the following examples. It should be understood, however, that the description following is illustrative only and should not be taken in any way as a restriction on the generality of the invention described above.

### EXAMPLES

### Example 1

### Immunoisolation of Viable ES Cells

This example details an immunomagnetic method for the purification of viable HES cells expressing the antigenic epitope detected by the monoclonal antibody GCTM-2. This procedure will enable efficient and repeatable studies of gene expression on antigenically defined ES cell populations. This procedure when incorporated into the current procedure for propagating HES cells will provide a rapid and reproducible methodology for the production of antigenically defined HES cells. In addition, this proposal details a method for colocalisation of antigens on an individual cell using double immunofluorescence techniques. Colocalisation of specific HES cell markers will enable identification of the phenotype of subpopulations of ES cells within the cultures and will help to isolate these subpopulations, which may have properties which are advantageous.

(i) Characterisation of Human ES Cells Using Double Immunofluorescence

Human ES (HES) cells were plated onto glass microscope slides and allowed to attach overnight at 37° C., 5% CO₂, after which they were fixed in ice-cold 100% ethanol. Slides were then air dried and stored at −20° C. until required. Slides were stained with mouse monoclonal antibodies raised against GCTM-2, TG-30, TG-343 (this laboratory), TRA 1-60, (a gift of Peter Andrews, University of Sheffield) and Oct-4 (Santa Cruz Biotechnology, Santa Cruz Calif.). GCTM-2, TG-343 and TRA 1-60 all detect epitopes on a keratan sulfate/chondroitin sulfate pericellular matrix proteoglycan found on embryocarcinoma (EC) and HES cells. GCTM-2 and TG-343 detect an epitope on the protein core of the proteoglycan while TRA 1-60 most likely binds to the O-linked lactosaminoglycan side chain attached to the protein core (Badcock et al., 1999; Cooper et al., 1992). Oct-4 is a POU-domain transcription factor that is required for formation of pluripotent stem cells in the mammalian embryo (Nichols et al., 1998). It is down regulated during gastrulation, eventually becoming restricted to the germ line and is used as a marker of undifferentiated pluripotent stem cells in vitro. The antigenic epitopes detected by GCTM-2, TG-343, TRA 1-60 and Oct-4 all disappear upon differentiation of HES cells (Reubinoff et al., 2000). These primary antibodies were incubated with HES cells fixed onto glass microscope slides with 100% ethanol.

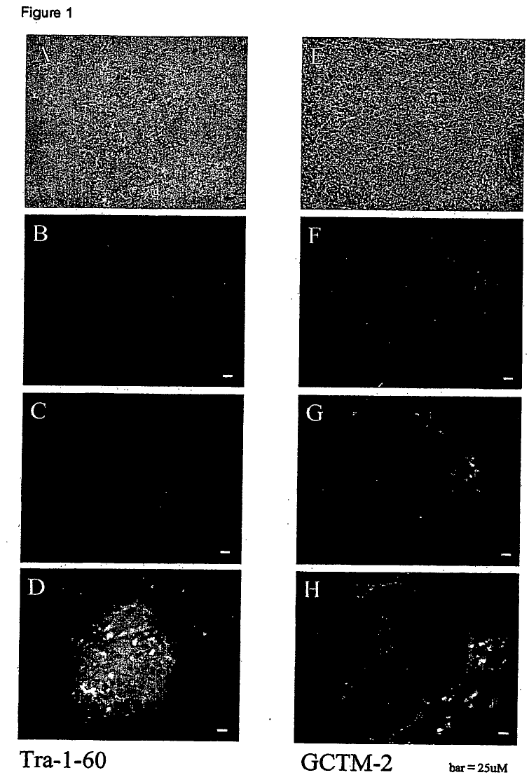

Distinct, double immunofluorescence staining was achieved by utilising an anti-mouse IgM specific antibody conjugated to biotin (Dako, Carpinteria, Calif.) and detected in turn by streptavidin conjugated to Texas Red (Amersham, Arlington Heights, Ill.) in conjunction with a rabbit anti-mouse Ig conjugated to fluorescein isothiocyanate (FITC; Dako). For example the staining achieved in FIG. 1(A-D) was achieved by incubating HES cells with Oct-4 (30 mins at RT) followed by washes with PBS (extensive PBS washes were carried out between each step), incubation with anti-mouse Ig-FITC (30 mins at RT), blocking with normal mouse Ig (30 mins at RT), incubation with TRA 1-60 (30 mins at RT), incubation with anti-mouse IgM-biotin (30 mins at RT), incubation with streptavidin-Texas Red (30 mins at RT), and finally with Hoechst dye (10 mins at RT). After rinsing with PBS, slides were mounted in Vectashield (Vector Laboratories, Burlingame, Calif.) and then either viewed under brightfield or with epifluorescence optics. Similar results were achieved using GCTM-2 as the second primary antibody (FIG. 1, E-H).

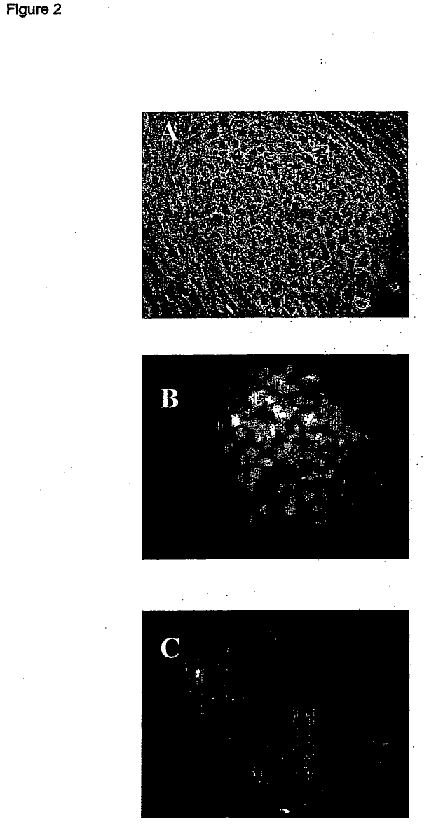

FIG. 2 shows double immunofluorescent staining with Oct-4 and TG-343. HES cells (FIG. 2A) were incubated with the Oct-4 antibody detected by an anti-mouse Ig-FITC secondary antibody (FIG. 2B). FIG. 2C shows the same field as A and B but showing fluorscent staining of the TG-343 antibody detected by an anti IgM-biotin-streptavidin-Texas Red complex.

(ii) Coating of Immunomagnetic Beads with GCTM-2

750 μg (25 μl) of rat anti-mouse IgM beads (Dynal, Oslo, Norway) were washed in phosphate buffered saline (PBS). PBS was removed from beads using the Dynal magnetic particle concentrator (MPC). GCTM-2 (500 μl) was then added to the beads at room temperature (RT) for 30 minutes. Unbound GCTM-2 is removed from the beads with 3 PBS washes utilising the MPC, followed by three washes in binding media (Dulbeccos modified Eagle medium (DMEM, without sodium pyruvate with glucose 4500 mg/L, Life Technologies, Rockville, Md. USA) supplemented with 25 mM Hepes, foetal bovine serum (FBS, 1%, Hyclone, Utah, USA), 2 mM glutamine, 50 units ml⁻¹ penicillin, and 50 μg ml⁻¹ streptomycin (Life Technologies)). Beads were then resuspended in 50 μl of binding media.

(iii) Human ES Cell Preparation

Human ES (HES) cells were harvested seven days after transfer onto mitomycin C mitotically inactivated mouse embryonic fibroblast (MEF) feeder layer (MEFS were isolated from day 13.5 post coitum foetuses of either the 129/Sv strain or F1 crosses of this strain with C57/BL6; used at 75,000 cells cm⁻²) in gelatin-coated tissue culture dishes. HES culture medium consisted of Dulbecco's modified Eagle medium (DMEM, without sodium pyruvate, glucose 4500 mg L⁻¹; Life Technologies, Rockville, Md.) supplemented with 20% foetal bovine serum (FBS; Hyclone, Logan, Utah), 0.1 mM β-mercaptoethanol, 1% nonessential amino acids, 2 mM glutamine, 50 units ml⁻¹ penicillin, and 50 mg ml⁻¹ streptomycin (Life Technologies). HES cell colonies were removed from the MEF feeder layer by exposure to dispase (10 mg ml⁻¹; Life Technologies). HES colonies were then washed twice in PBS without calcium or magnesium and gently disassociated using a 200 μl pipette tip. HES cells were then centrifuged (4 min, 0.2 g), supernatants carefully removed and resuspended in binding medium. A small aliquot of cells (starting material) was saved for immunofluorescent analysis.

(iv) Immunomagnetic Isolation of GCTM-2 Positive HES Cells.

Cells were resuspended in binding medium and added to washed, GCTM-2 coated beads (see above). The cell-bead mixture was incubated on ice for 30 minutes with frequent mixing. A microfuge tube containing cell-bead mixture was placed in Dynal MPC for 1 minute, and supernatant was removed and saved as unbound fraction. The cell-bead mixture was rinsed twice more in binding medium and supernatants were also kept as unbound fraction. Cells remaining on beads were resuspended in binding medium and kept as bound fraction. Starting material, bound and unbound fractions were plated onto 6 well tissue culture plates or 8 chamber slides (Lab-Tek, Nunc,. Naperville, Ill.) and incubated at 37° C., 5% CO₂ in a humidified chamber for subsequent morphological and immunofluorescent analysis. Cells were, also analysed by RT-PCR (see below). Cells plated on the 8 chamber slides for immunofluorescent analyses were incubated overnight at 37° C. prior to fixation in ice-cold 100% ethanol. Slides were then air dried and stored at −20° C. until required.

(v) Immunofluorescent Characterisation of Immunomagnetically Purified HES Cells.

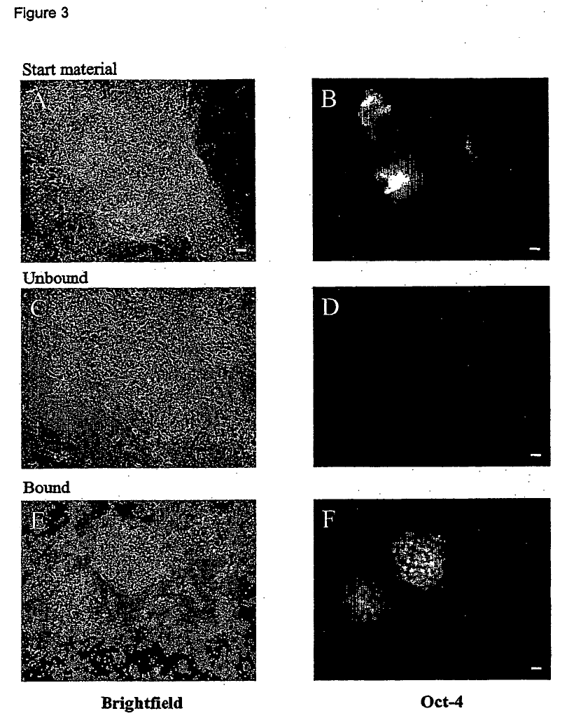

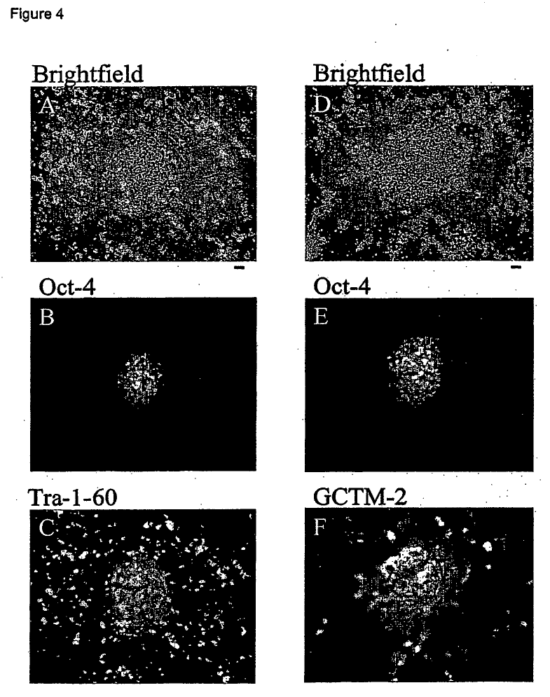

Starting material, bound and unbound fractions were stained with mouse monoclonal antibodies raised against GCTM-2, TRA 1-60 and Oct-4. (FIG. 3 and FIG. 4). Antibody localization was performed by using rabbit anti-mouse immunoglobulins conjugated to fluorescein isothiocyanate (FITC; Dako, Carpinteria, Calif.). After rinsing, slides were mounted in Vectashield (Vector Laboratories, Burlingame, Calif.) and then either viewed under brightfield or with epifluorescence optics.

(vi) Isolation of mRNA and RT-PCR Analysis of Gene Expression.

Bound and unbound HES cells were isolated as above and lysed in 300 μl lysis/binding buffer (Dynal AS, Oslo), Following cell lysis, mRNA was isolated by incubation with oligo(dT) bound dynabeads (Dynal). Bound mRNA was washed twice by resuspending in a lithium dodecylsulphate (LIDS) containing washing buffer and removal of supernatants using the MPC (Dynal). Three further washes with wash buffer without LIDS were carried out and dynabeads resuspended in 100 μl wash buffer. Reverse transcription was carried out using Superscript reverse transcriptase II (Life Technologies) following the manufacturers instructions. PCR for specific mRNAs was performed using 35 cycles of 95° C. for 1 min, 55° C. for 2 min and 72° C. for 2 min, followed by a final incubation at 72° C. for 6 min. PCR primers were synthesized by Pacific Oligos (Adelaide, Australia). Oct-4 transcripts were assayed using the following primers:

5′-CGTTCTCTTTGGAAAGGTGTTC (forward)
and
3′-CACTCGGACCACGTCTTTC (reverse).

As a control for mRNA quality, β-actin transcripts were assayed using the same RT-PCR and the following primers: 5′-CGCACCACTGGCATTGTCAT-3′ (forward), 5′-TTCTCCTTGATGTCACGCAC-3′ reverse). Amplified products were resolved by agarose gel electrophoresis and visualised after ethidium bromide staining.

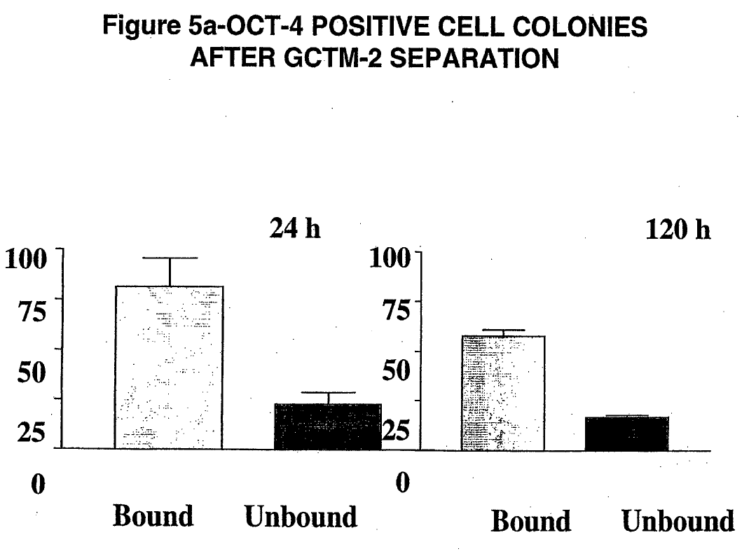

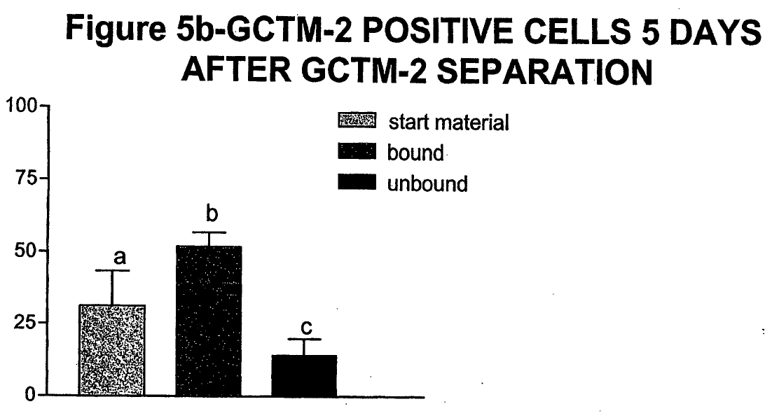

FIG. 1 shows double label immunostaining for Oct-4, TRA1-60 and GCTM-2 on human ES cell cultures. It is clear that there is significant overlap between the Oct-4 positive subpopulation, and the cells expressing the epitopes detected by the latter two antibodies. However, some Oct-4 positive cells are not labelled with either GCTM-2 or TRA1-60. FIG. 3 shows that the immunoisolation procedure greatly enriches for clusters of cells which are positive for Oct-4, and that these cells are viable and able to reattach to the monolayer after overnight culture. It is of interest that although probably not all cells in a cluster are accessible to the antibody-bead complex, it appears that all cells in bound clusters are positive for Oct-4, and for GCTM-2 and TRA1-60. This indicates that stem cells positive for GCTM-2 probably grow in coherent clusters, consistent with immunostaining for this marker. Cells isolated as shown in FIGS. 3 and 4 may be serially cultivated and maintain stem cell properties. This is shown in FIG. 5A which shows the isolated cells maintain stem cell characteristics of Oct-4 expression after 24 hours and 120 hours compared with the unbound population. After separation of HES cells with GCTM-2 complexed to magnetic beads there is a large enrichment for Oct-4 positive cell colonies in the fraction bound to GCTM-2 (Bound) as compared to the fraction of cells that did not bind to the GCTM-2 magnetic beads (Unbound). This is true for both 24 hours (left hand panel, Bound =˜80% positive vs Unbound=˜20% positive) and 120 hours (right hand panel, Bound=˜60% positive vs Unbound=|15% positive) after separation. These results imply that separation for GCTM-2 positive cells also enriches for other stem cell markers.

FIG. 5B shows that after 120 hours (5 days) the cells are also positive for GCTM-2 with a significant enrichment for GCTM-2 positive cells in the bound fraction as compared to the starting material and unbound fractions (p<0.05. These results were obtained after GCTM-2 separation. Cells were cultured on 8 chamber slides with MEFS for 5 days. Cells from each fraction were removed by dispase, trypsinized to a single cell suspension, stained in solution for GCTM-2 detected by FITC immunofluorescence and counted under the fluorescence microscope (n=5 separate slides).

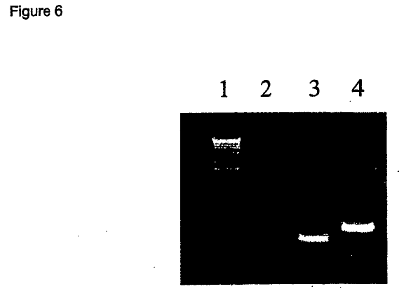

FIG. 6 confirms that the isolated cell populations express Oct-4 transcripts.

The immunoisolation procedure described above provides for the isolation of a viable pure population of GCTM-2⁺, Oct-4⁺ cells from routinely sub-cultured populations of human ES cells. The procedure will be useful in analysis of gene expression in ES cultures, thereby providing results on a defined population of cells without contamination from minority populations of differentiated cells. By eliminating the differentiated cells, the growth of the stem cells may be improved. The procedure, unlike those based on flow cytometry, does not rely on dissociation of the ES cultures to single cells and thereby ensures good viability of the isolated cells. It should be possible to incorporate this procedure into routine subculture methodology to replace the technique of isolation of stem cells under microscopic control used presently to eliminate differentiated cells, thereby facilitating culture scaleup.

### Example 2

### Identifying a Novel HES Cell Subpopulation

Cells were isolated from differentiating human embryonic stem (ES) cell colonies (HES-2) six weeks following plating and transferred onto a Mitomycin C (0.5 mg/ml; Bristol-Myers Squibb, Australia) treated mouse embryonic fibroblast cell line (STO) cultured in DMEM medium (Gibco, without sodium pyruvate, with glucose 4500 mg/L) supplemented with 20% (v/v) Fetal Bovine Serum (FBS; Hyclone, Utah), β-mercaptoethanol (0.1 mM; GIBCO), 1% (v/v) Non Essential Amino Acids (NEM; GIBCO), 1% (v/v) insulin, selenium and transferrin solution (ITS, GIBCO), 50 ng/ml basic fibroblast growth factor (b-FGF, R and D Systems), glutamine (2 mM; GIBCO), penicillin and steptomyocin (50 μg/ml; GIBCO).

From seven days after plating the stem cell colonies were further propagated by mechanical isolation of the cells from the surrounding areas of differentiating cells. This technique involved aspiration of the culture medium and washing of the cells with phosphate-buffered saline (PBS: Gibco) containing calcium and magnesium. The stem cell colonies were mechanically cut using a heat pulled glass micro-pipette. The PBS was then removed and pre-equilibrated culture medium containing dispase (10 mg/ml; GIBCO) added. Following a three-minute incubation detached clumps of stem cells were washed twice in PBS (Gibco) and transferred to a fresh STO feeder layer.

(i) Cell Characterisation

Characterization of Cells Using Immunfluorescence Microscopy

Cells were transferred onto 8-well chamber slides (Lab-Tek) and cultured for seven to ten days. Following two washes with warmed PBS the cells were fixed with cold 80% (v/v) Ethanol and air-dried.

(ii) Isolation of mRNA and RT-PCR Analysis of Gene Expression

Stem cell-like cultures were isolated and lysed in 300 μl lysis/binding buffer (Dynal AS, Oslo). Following cell lysis, mRNA was isolated by incubation with oligo(dt) bound Dynabeads (Dynal). Bound mRNA was washed twice by resuspension in a lithium-sodiumdodecylsulphate (LIDS) containing wash buffer (100 mM Tris-HCl, 500 mM Lithium Chloride, 10 mM EDTA, 5 mM dithiothreitol) and removal of the supernatant achieved by a magnet. Three further three washes with wash buffer without LIDS were carried out and Dynabeads resuspended in 100 μl wash buffer. Reverse transcription was carried out using Superscript reverse transcriptase II (Life Technologies) following the manufacturer's protocol. Polymerase Chain Reaction for specific mRNAs was performed using 35 cycles of 94° C. for 1 min, 55° for 2 min, and 72° C. for 2 min, followed by a final incubation of 72° C. for 6 min. PCR primers (Table 1) were synthesized by Pacific Oligos (Adelaide, Australia) or Sigma. Amplified products were resolved by agarose gel electrophoresis and visualized after ethidium bromide staining.

| | | |
|---|---|---|
| | Forward Primer | Reverse Primer |
| Oct 4 | 5′-CGT TCT CTT TGG AAA GGT GTT C-3′ | 5′-ACA CTC GGA CCA CGT CTT TC-3′ |
| CR-1 | 5′-CAG AAC CTG CTG CCT GAA TG-3′ | 5′-GTA GAA ATG CCT GAG GAA ACG-3′ |
| Genesis | 5′ | |
| HNF3α | 5′-GAG TTT ACA GGC TTG TGG CA-3′ | 5′-GAG GGC AAT TCC TGA GGA TT-3′ |
| AFP | 5′-CCA TGT ACA TGA GCA CTG TTG′3′ | 5′-CTC CAA TAA CTC CTG GTA TCC-3′ |
| Sox 17 | 5′-CGC ACG GAA TTT GAA GAG TA-3′ | 5′-GGA TCA GGG AGC TGT GAC AG-3′ |
| Nestin | 5′-GAG CTG GCG GAC CTC AAG ATG-3′ | 5′-AGG GAA GTT GGG CTC AGG ACT GG-3′ |
| Pax 6 | 5′-AACAGACACAGCGCTCAGAAAGA-3′ | 5′-CGGGAACTTGAAGTGGAACTGAC-3′ |
| Vitronectin | 5′-TTG GAG GCA CTC AGC TAG AA-3′ | 5′-TGT TCA TGG AGA GTG GCA TT-3′ |
| Transferrin | 5′-GTG AGC TGA GCT GGG ACA AT-3′ | 5′CCA TCA AGG CAG AGC AAC TG-3′ |
| Actin | 5′-GGCAGGACTGGGATTGTCAT-3′ | 5′-TTCTCCTTGATCGTCACGCAC-3′ |

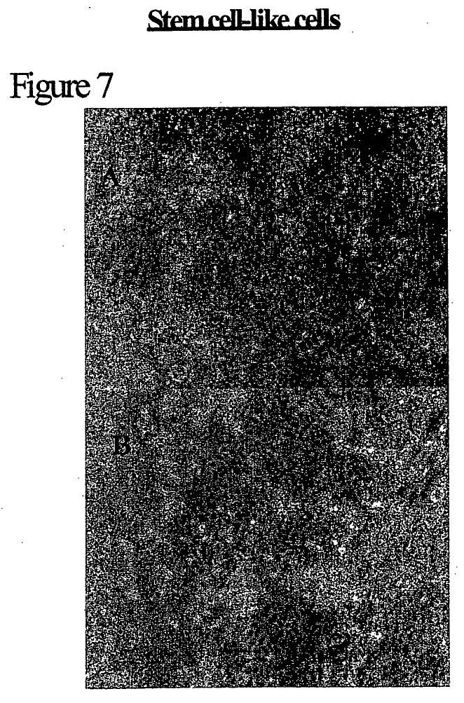

Twenty-four hours following transfer of cells from six week old dishes of human ES cells, clusters of small flat cells appeared outgrowing from cell clumps. Within a further two to three days rounded cell colonies formed. Under phase contrast microscopy, cells with a distinct morphological appearance could be identified with a translucent cytoplasm and darkened nucleus (FIG. 7A). After a further seven to ten days in culture other cell types appear within the colony as cells begin to differentiate (FIG. 7B). Most commonly, but not invariably, an area of differentiated cells appear within the centre of the stem cell-like colony. Over a period of two- to three weeks, the cells differentiated into a variety of cell types including early neural and epithelial cell types and cells similar to extraembryonic cell types with a squamous appearance and an association with cystic structures (Pera et al., 2001).

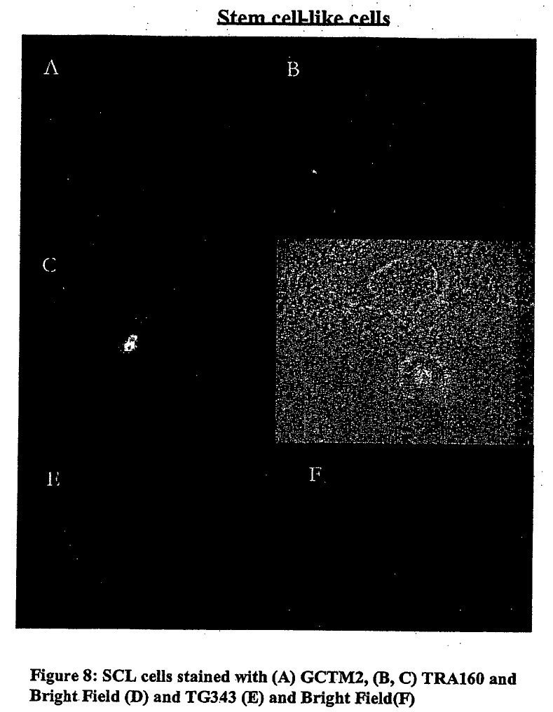

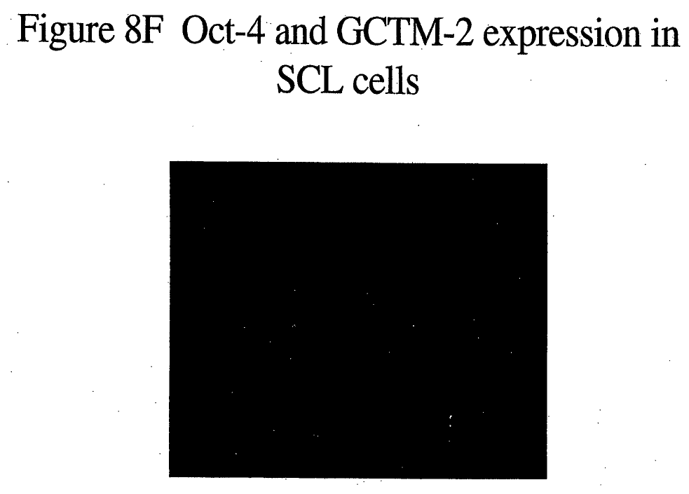

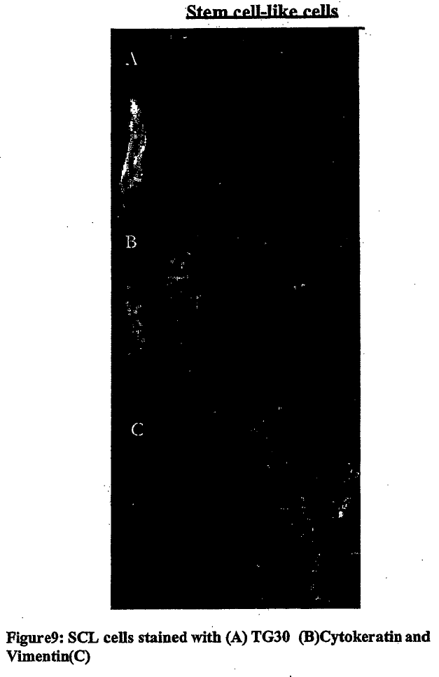

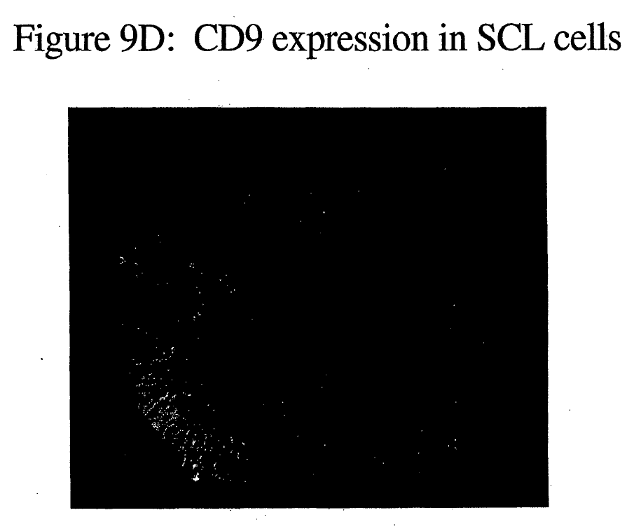

Indirect immunofluorescence microscopy of the SCL cells showed negative staining for GCTM2 (FIG. 8A), TRA-160 (FIGS. 8B-D), TG343 (FIG. 8E) and SSEA4 (data not shown). The cells displayed positive fluorescent staining for TG30 (FIG. 9A) and antibody CAM 5.2 against low molecular weight cytokeratins (FIG. 9B) as well as a low intensity fluorescence for the intermediate filament marker Vimentin (FIG. 9C). No positive fluorescence was observed for SSEA-1, PHM4 (HLA antigen) and the epithelial marker EpCAM (clone Ber-EP4; DAKO).

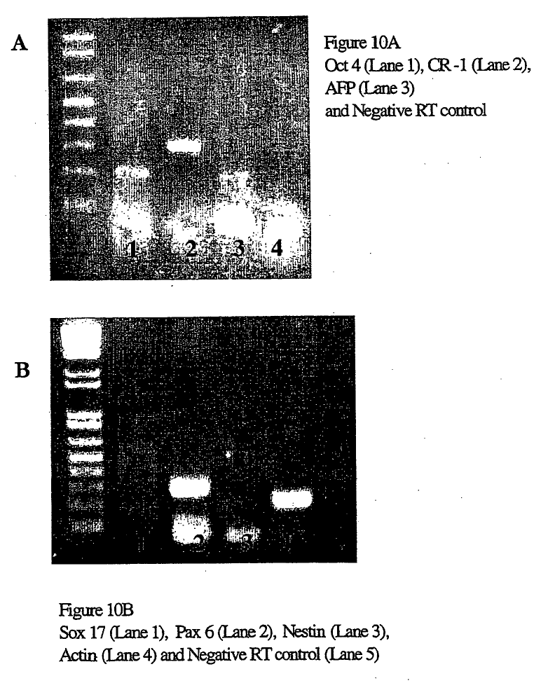

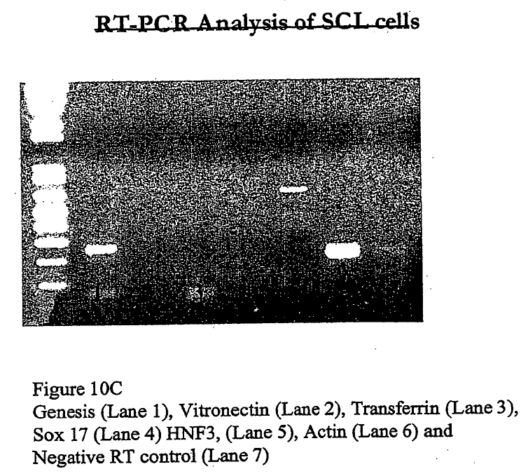

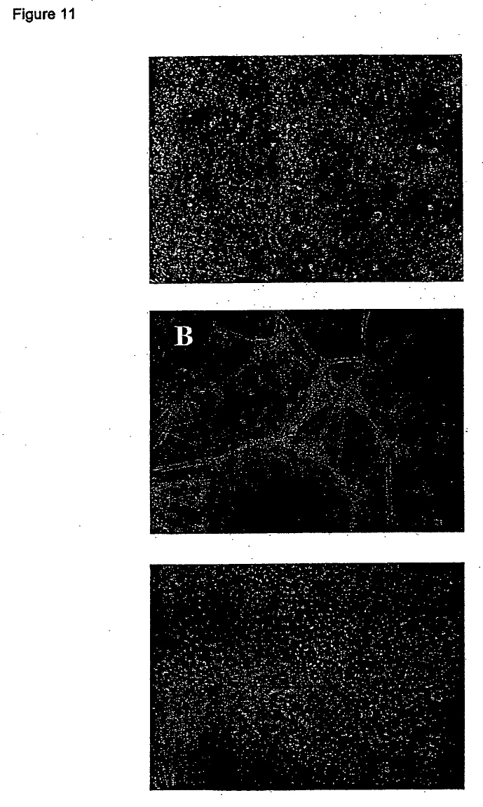

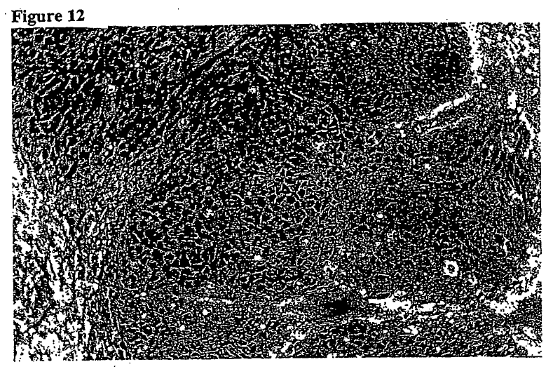

Expression of genes known to be transcriptionally active within pluripotent stem cell populations including Oct-4, Cripto (FIG. 10A) and Genesis (FIG. 10C). Transcripts for the neural marker Pax 6 (FIG. 10B) and relatively low intensity transcripts for hepatocyte nuclear factor (HNF)-3α (FIG. 10C) were also detected, as they are in conventional ES cell cultures. There were no transcripts for the endoderm genes alphafetoprotein, vitronectin, or transferrin, nor for the neural marker nestin, although verification of successful RT-PCR was validated by identification of the housekeeping gene actin.

The cell population described here clearly differs from the canonical primate pluripotent stem cell in that it lacks expression of the surface markers usually found on ES cells. Nevertheless, this cell type expresses other markers of human ES cells and undergoes differentiation at the morphological level into a range of cell types. Thus it is possible that the SCL cell is a novel type of pluripotent cell which derives from, or is a precursor of, the canonical ES cell. The SCL cell may have desirable properties compared to conventional ES cells, such as ease of growth during routine subculture or cloning, or more extensive capacity for in vitro differentiation into diverse lineages, or greater susceptibility to uptake and integration of DNA constructs. For example, SCL cells may be maintained on STO cells, a fibroblast cell line which is easy to grow and maintain, rather than on cultures of mouse embryo fibroblasts.

SCL cells (FIG. 11A) were maintained in a similar manner as HES cells and were capable of differentiating into a number of cell types including neurospheres (FIG. 11B), and epithelial-like cells (FIG. 11C).

### Example 3

### A Differentiated Epithelial Stem Cell from Human ES Cell Cultures: Putative Endodermal Progenitor

Epithelial cells, identified morphologically, were isolated from differentiating human embryonic stem (ES) cell colonies (HES2) and stem-cell like (SCL) colonies (Example 2 above, Hawes and Pera; 2001), four to eight weeks following subculture. Alternately, these cells could be induced to appear in cultures of human ES cells using the following methodology. ES cells were maintained for 4-10 days under standard conditions, then the culture medium was switched to DMEM, low glucose formulation with sodium pyruvate, supplemented with reduced levels of fetal calf serum (5-10%), and 10 mm nicotinamide. Within 7-14 days, the predominant cell type in the culture was an epithelial cell similar to those illustrated in FIG. 11C. The large, flat, squamous cells found in control cultures under suboptimal conditions (eg low serum without nicotinamide) were much less frequent in the presence of nicotinamide. After spontaneous appearance or induction as described above, cells were transferred onto a Mitomycin C (0.5 mg/ml; Bristol-Myers Squibb, Australia) treated mouse embryonic fibroblast cell line (STO) cultured in DMEM medium (Gibco, without sodium pyruvate, with glucose 4500 mg/L) supplemented with 20% (v/v) Fetal Bovine Serum (FBS; Hyclone, Utah), β-mercaptoethanol (0.1 mM; GIBCO), 1% (v/v) Non Essential Amino Acids (NEAA; GIBCO), 1% (v/v) insulin, selenium and transferrin solution (ITS, GIBCO), glutamine (2 mM; GIBCO), penicillin and steptomyocin (50 μg/ml; GIBCO). In some cases, the culture medium was supplemented with Cholera Toxin (0.02 μg/μl; Sigma) or Stem Cell Factor (SCF, 25 ng/μl; Peprotech).

Six to eight weeks after plating the epithelial-like cell colonies were further propagated by mechanically cutting of sheets of cells into smaller clumps. This technique involved aspiration of the culture medium and washing of the cells with phosphate-buffered saline (PBS; Gibco) containing calcium and magnesium.

(i) Characterization of Cells Using Immunofluorescence Microscopy

Cells were transferred onto 8-well chamber slides (LaboTek) and cultured for up to four weeks until colonies with their distinctive appearance appeared. Following two washes with warmed PBS the cells were fixed with cold 80% (v/v) Ethanol and air-dried. Staining was carried out as described above.

(ii) Isolation of mRNA and RT-PCR Analysis of Gene Expression

Epithelial-like cell colonies were isolated (described above) and transferred into 300 μl lysis/binding buffer (Dynal AS, Oslo). Following cell lysis, mRNA was isolated by incubation with oligo(dT) bound dynabeads (Dynal). Bound mRNA was washed twice by resuspension in a lithium-sodiumdodecyl sulphate (LIDS) containing wash buffer (100 mM Tris-HCl, 500 mM Lithium Chloride, 10 mM EDTA, 5 mM dithiothreitol) and removal of the supernatant using a magnet. Three further three washes with wash buffer without LIDs were carried out and dynabeads resuspended in 100 μl wash buffer. Reverse transcription was carried out using Superscript reverse transcriptase II (Life Technologies) following the manufacturer's protocol. Polymerase Chain Reaction for specific mRNAs was performed using 35 cycles of 94° C. for 1 min, 55° for 2 min, and 72° C. for 2 min, followed by a final incubation of 72° C. for 6 min. PCR primers were synthesized by Pacific Oligos (Adelaide, Australia) or Sigma. Amplified products were resolved by agarose gel electrophoresis and visualized after ethidium bromide staining.

| | | |
|---|---|---|
| | Forward Primer | Reverse Primer |
| Oct 4 | 5′-CGT TCT CTT TGG AAA GGT GTT C-3′ | 5′- ACA CTC GGA CCA CGT CTT TC-3′ |
| CR-1 | 5′- CAG AAC CTG CTG CCT GAA TG-3′ | 5′- GTA GAA ATG CCT GAG GAA ACG-3′ |
| HNF3α | 5′- GAG TTT ACA GGC TTG TGG CA-3′ | 5′- GAG GGC AAT TCC TGA GGA TT-3′ |
| AFP | 5′- CCA TGT ACA TGA GCA CTG TTG′3′ | 5′- CTC CAA TAA GTC CTG GTA TCC-3′ |
| Sox 17 | 5′- CGG ACG GAA TTT GAA CAG TA-3′ | 5′- GGA TCA GGG ACC TGT CAC AC-3′ |
| Nestin | 5′-CAG CTG GCG CAC CTC AAG ATG-3′ | 5′-AGG GAA GTT GGG CTC AGG ACT GG-3′ |
| Pax 6 | 5′-AACAGACACAGCCCTCACAAACA-3′ | 5′-CGGGAACTTGAACTGGAACTGAC-3′ |
| Vitronectin | 5′-TTG CAG GCA CTC AGC TAG AA-3′ | 5′-TGT TCA TGG ACA GTG GCA TT-3′ |
| Transferrin | 5′-CTG ACC TCA CCT GGG ACA AT-3′ | 5′CCA TCA AGG CAC AGC AAC TC-3′ |
| Actin | 5′-CGCACCACTGGCATTGTCAT-3′ | 5′-TTCTCCTTGATCGTCACGCAC-3′ |

Twenty-four hours following transfer, clusters of small flat cells appeared outgrowing from cell clumps. Within a further two to three days rounded cell colonies formed. Under light microscopy examination the cells had a distinctive morphological appearance with darkened cytoplasm and translucent nucleus (FIG. 12). Thin, fibroblastic cells typically surround the epithelial-like cell colonies. Some colonies were associated with a more rounded cell that detached from the epithelial-like cells as proliferation continued. Cell growth was definitely enhanced by addition of cholera toxin to the culture medium

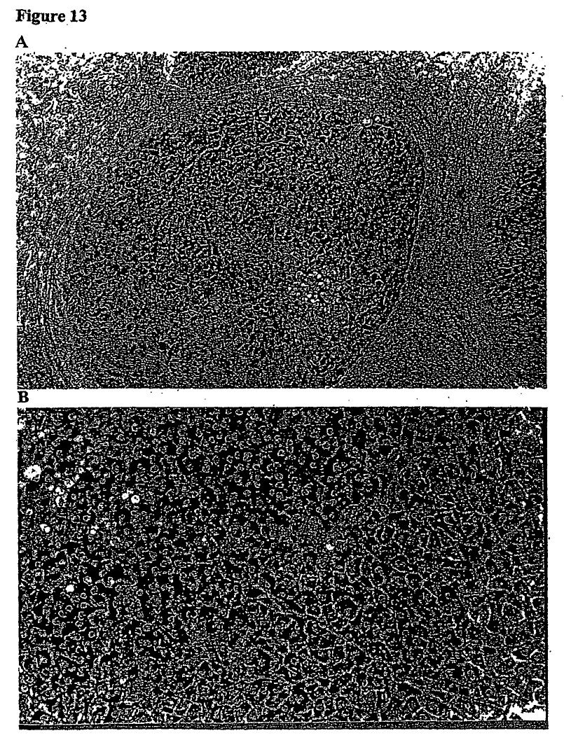

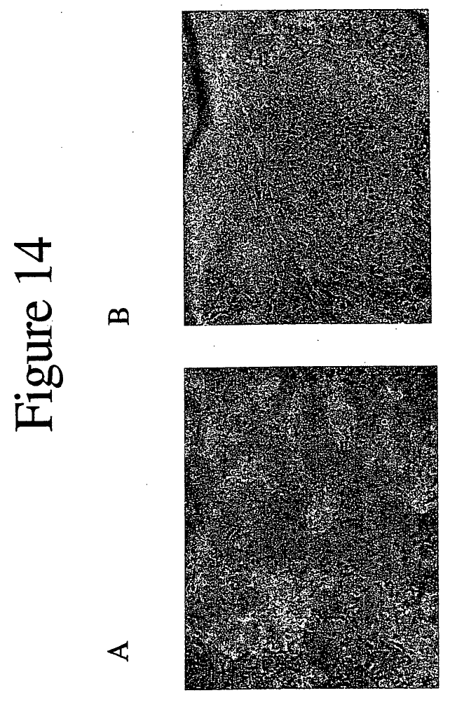

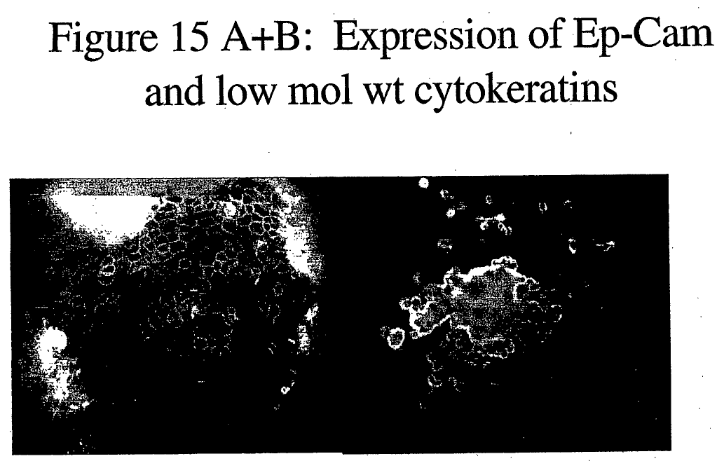

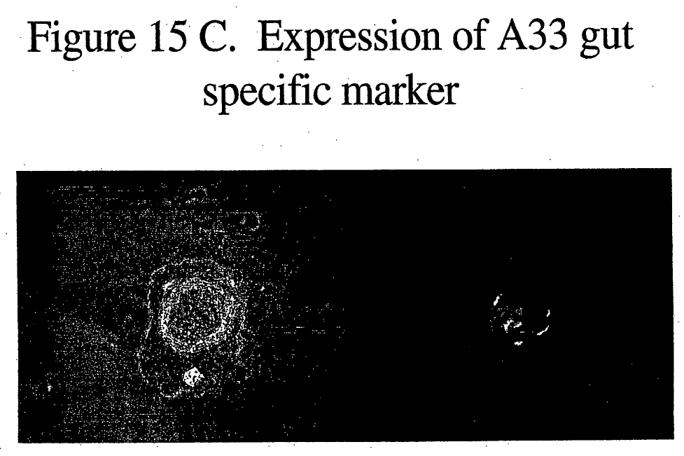

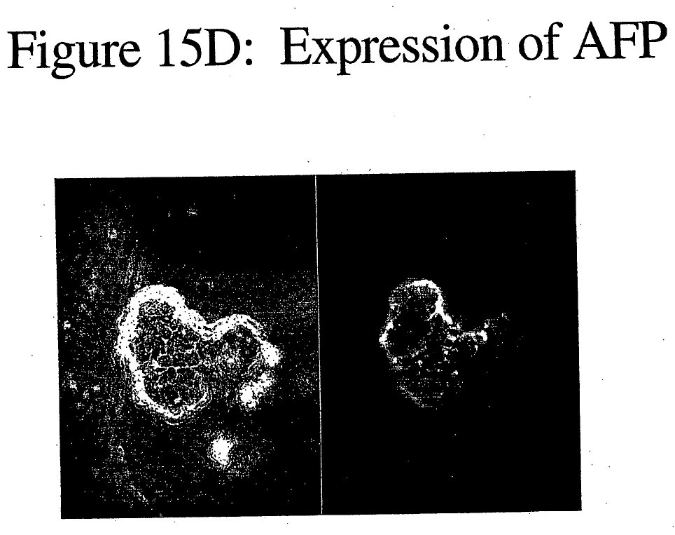

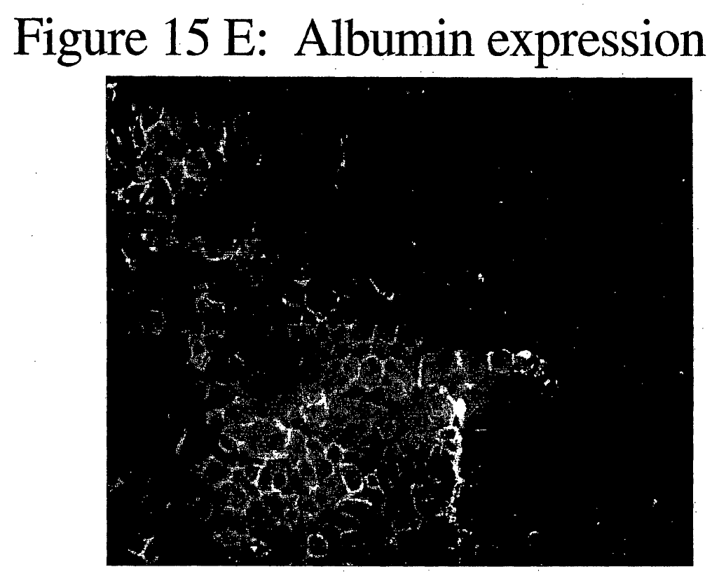

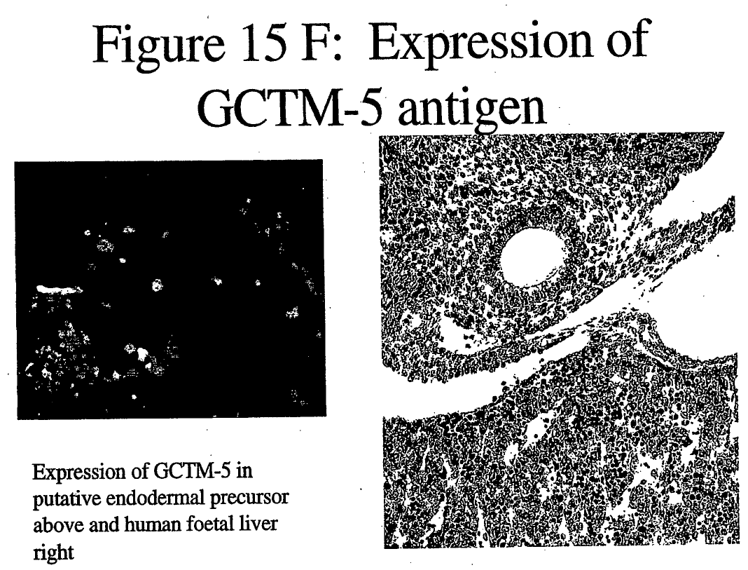

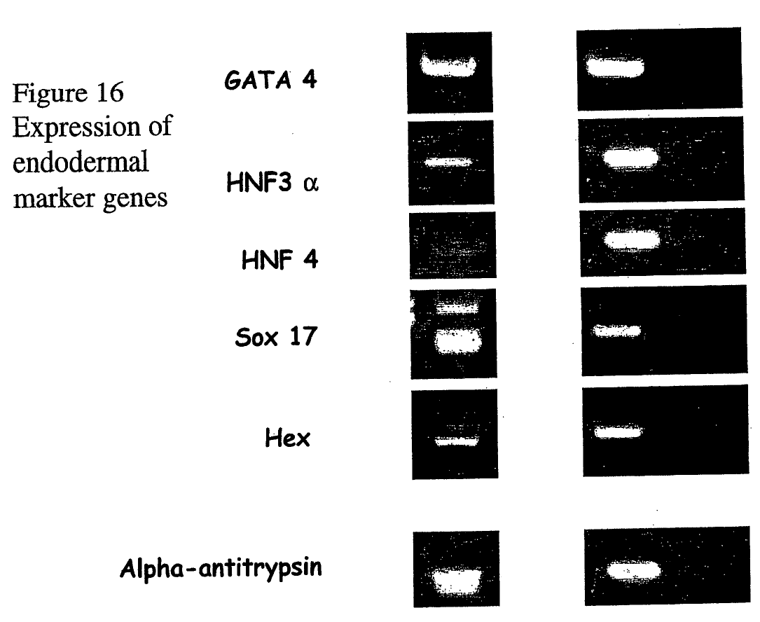

The epithelial cell colonies were mechanically cut using a heat pulled glass micro-pipette and transferred to a fresh STO feeder layer (FIG. 13A). It was also shown that cell colonies could be propagated on a thin Matrigel (Gibco) coat and retain their distinct morphology for seven to ten days (FIG. 13B). Following this fibroblast type cells began to appear at the edge of the colonies that necessitates their transfer to fresh Matrigel-coated dishes to maintain their specific phenotype.

Indirect immunofluorescence methodology showed positive staining for the epithelial cell markers EpCam (clone Ber-EP4; DAKO), and low molecular weight cytokeratins (Cam 52; Becton-Dickenson); Isolated patches of cells above the plane of the monolayer showed expression of the endoderm marker alphafetoprotein (AFP; DAKO) and the gut cell specific A33 antibody. No significant staining was observed for PHM4 (HLA Class I antigen), CD34 or the stem cell marker Oct-4 (Santa Cruz Biotechnologies).

The expression of hepatocyte nuclear factor (HNF)-3α, and HNF-4 was demonstrated by RT-PCR analysis. No evidence of gene-expression for Oct-4 or CR-1, characteristic of pluripotent stem cell populations was observed, although verification of successful RT-PCR was validated by identification of the housekeeping gene actin. Transcripts for Pax 6 were also observed.

Thus the novel cells are clearly epithelial in nature. They are no longer pluripotent stem cells; they lack all markers of pluripotent human stem cells and fail to show the typical multilineage differentiation shown by ES cells in vitro. The response of this cell to cholera toxin and its requirement for STO cells further distinguish it from human ES cells which are not affected by cholera toxin and which do not grow well on STO monolayers. The expression in some colonies of the markers AFP (made by embryonic endodermal tissue) and the A33 antigen (restricted to gut, Abud et al., 2000), as well as expression of HNF3 alpha and HNF-4, suggest that these primitive epithelial cells may be capable of differentiation into several embryonic endodermal derivatives. The cells may respond to various known inducers of endodermal differentiation by forming different derivatives such as gut, liver lung and pancreas. They may provide an in vitro source of these important cell types.

### References

Abud, H. E., Johnstone, C. N., Tebbutt, N. C. and Heath, J. K. (2000). The murine A33 antigen is expressed at two distinct sites during development, the ICM of the blastocyst and the intestinal epithelium. Mech Dev 98, 111-4.

Badcock, G., Pigott, C., Goepel, J. and Andrews, P. W. (1999). The human embryonal carcinoma marker antigen TRA-1-60 is a sialylated keratan sulfate proteoglycan. Cancer Res 59, 4715-9.

Cooper, S., Pera, M. F., Bennett, W. and Finch, J. T. (1992). A novel keratan sulphate proteoglycan from a human embryonal carcinoma cell line. Biochem J 286, 959-66.

Gualdi, R., Bossard, P., Zheng, M., Hamada, Y., Coleman, J. R. and Zaret, K. S. (1996). Hepatic specification of the gut endoderm in vitro: cell signaling and transcriptional control. Genes Dev 10, 1670-82.

Nichols, J., Zevnik, B., Anastassiadis, K., Niwa, H., Klewe-Nebenius, D., Chambers, I., Scholer, H. and Smith, A. (1998). Formation of pluripotent stem cells in the mammalian embryo depends on the POU transcription factor Oct4. Cell 95, 379-91.

Pera, M. F., Reubinoff, B. and Trounson, A. (2000). Human embryonic stem cells. J Cell Sci 113, 5-10.

Reubinoff, B. E., Pera, M. F., Fong, C. Y., Trounson, A. and Bongso, A. (2000). Embryonic stem cell lines from human blastocysts: somatic differentiation in vitro. Nat Biotechnol 18, 399-404.

Saloman, D. S., Bianco, C., Ebert, A. D., Khan, N. I., De Santis, M., Normanno, N., Wechselberger, C., Seno, M., Williams, K., Sanicola, M. et al. (2000). The EGF-CFC family: novel epidermal growth factor-related proteins in development and cancer. Endocr Relat Cancer 7, 199-226.

Sutton, J., Costa, R., Klug, M., Field, L., Xu, D., Largaespada, D. A., Fletcher, C. F., Jenkins, N. A., Copeland, N. G., Klemsz, M. et al. (1996). Genesis, a winged helix transcriptional repressor with expression restricted to embryonic stem cells. J Biol Chem 271, 23126-33.

Finally it is to be understood that various other modifications and/or alterations may be made without departing from the spirit of the present invention as outlined herein.

1. A method of identifying a viable sub-population of HES cells, said method comprising:
obtaining a source of HES cells; and
identifying the sub-population of HES cells that are at least GCTM-2 positive.

2. A method according to claim 1 wherein the sub-population of HES cells are identified by expression of GCTM-2 antigens and expresses at least one other surface antigen of ES cells.

3. A method according to claim 1 or 2 wherein the sub-population of HES cells are undifferentiated or pluripotent HES cells.

4. A method according to claim 1 wherein the source of HES cells is derived from an embryo or a culture of HES cells.

5. A method according to claim 2 wherein said at least one other surface antigen is Oct-4.

6. A method according to claim 2 wherein said at least one other surface antigen is TRA1-60, TG30 or CD9.

7. An undifferentiated or pluripotent HES sub-population identified by a method according to any one of claims 1 to 2 or 4 to 6.

8. A method of isolating a viable sub-population of HES cells, said method comprising:
obtaining a source of HES cells;
identifying HES cells that are at least GCTM-2 positive; and
selecting for or against those cells which are GCTM-2 positive.

9. A method of isolating a viable sub-population of HES cells, said method comprising:
obtaining a source of HES cells;
exposing the cells to a means that specifically recognizes GCTM-2;
reacting the cells to the means to bind the sub-population of cells to the means; and
separating the sub-population of cells which are bound from the unbound cells.

10. A method according to claim 8 or 9 wherein the sub-population of HES cells are undifferentiated or pluripotent HES cells.

11. A method according to claim 10 wherein the source of HES cells is derived from an embryo or a culture of HES cells.

12. A method according to claim 8 or 9 wherein the subpopulation of HES cells is additionally Oct-4 positive.

13. A method according to claim 8 or 9 wherein the subpopulation of HES cells is additionally TRA1-60, TG30 or CD9 marker positive.

14. A method according to claim 9 wherein the means is a support coupled to an antibody for GCTM-2.

15. A method according to claim 14 wherein the support is additionally coupled to a means that specifically recognizes TRA1-60, CD9 or TG30 or other surface markers of ES cells.

16. A method according to claim 8 or 9 wherein the source of HES cells are dissociated to yield a suspension of cells in clumps of approximately 10 to 100 cells.

17. A method according to claim 9 wherein the separation of the sub-population of cells comprises sorting single cells by flow cytometry.

18. An isolated and viable sub-population of HES cells.

19. An isolated and viable undifferentiated or pluripotent sub-population of HES cells.

20. An isolated and viable sub-population of HES cells wherein said subpopulation is positive for GCTM-2.

21. An isolated and viable sub-population according to claim 20 that is additionally positive for Oct-4, TRA1-60, TG30 or CD9.

22. An isolated and viable sub-population according to claim 20 or 21 comprising undifferentiated or pluripotent HES cells.

23. An isolated and viable sub-population of HES cells according to claim 20 or 21, capable of sub-culture.

24. An isolated and viable sub-population according to claim 23 comprising undifferentiated or pluripotent cells.

25. An isolated and viable sub-population isolated by a method according to claim 8 or 9.

26. An isolated and viable differentiated and pluripotent sub-population of HES cells.

27. An isolated and viable sub-population of HES cells wherein said subpopulation is negative for GCTM-2.

28. An isolated and viable sub-population according to claim 27 that is additionally negative for TRA1-60.

29. An isolated and viable sub-population according to claim 27 or 28 comprising differentiated HES cells.

30. An isolated and viable sub-population of HES cells according to claim 27 or 28 capable of sub-culture.

31. A method of subculturing HES cells, said method comprising:
obtaining a source of HES cells;
exposing the cells to a means that specifically recognizes GCTM-2;
reacting the cells to the means to bind the sub-population of cells to the means;
separating sub-populations of HES cells which are bound or unbound to the means; and
sub-culturing the bound cells.

32. A method of subculturing HES cells, said method comprising:
obtaining a source of HES cells;
exposing the cells to a support coupled to at least one means that specifically recognizes for GCTM-2;
reacting the cells to the means to bind the sub-population of cells to the means;
separating bound and unbound sub-populations of HES cells; and
sub-culturing the bound cells.

33. A method according to claim 32 wherein the separation of bound and unbound HES cells comprises sorting single cells by flow cytometry.

34. A method according to claim 31 or 32 wherein the subpopulation of HES cells are undifferentiated or pluripotent HES cells.

35. A method according to claim 34 wherein the source of HES cells is derived from an embryo or a culture of HES cells.

36. A method according to claim 31 or 32 wherein the subpopulation of HES cells is additionally Oct-4 positive.

37. A method according to claim 31 or 32 wherein the subpopulation of HES cells is additionally TRA1-60, TG30 or CD9 marker positive.

38. A method according to claim 32 wherein the means is an antibody for GCTM-2.

39. A method according to claim 38 wherein the support is additionally coupled to a means that specifically recognizes TRA1-60, TG30 or CD9.

40. A sub-cultured sub-population of HES cells prepared by a method according to claim 31 or 32.

41. A method of transplantation in a patient, said method comprising:
obtaining a sub-population of HES cells according to any one of claims 19-21 or 26-28; and
transplanting the cells into said patient.

42. A method according to claim 41 wherein the HES cells are sub-cultured prior to transplanting into the patient.

43. A method according to claim 41 wherein the HES cells are undifferentiated or pluripotent cells.

44. A method according to claim 41 wherein the HES cells are differentiated cells.

45. A method of identifying gene expression in a HES cell, said method comprising:
obtaining a substantially homogenous HES cell sub-population; and
conducting gene expression analysis on the sub-population.

46. A method of identifying gene expression in a HES cell, said method comprising:
obtaining a substantially homogenous HES cell sub-population isolated by a method according to claim 8 or 9; and
conducting gene expression analysis on the sub-population.

47. A method according to claim 46 wherein the gene expression is identified during differentiation of HES cells.

48. A subpopulation of HES cells which are morphologically distinct from HES cells and have stem cell-like (SCL) characteristics, said sub-population of HES cells having the ability to develop into various cell types and wherein said cells have at least one of the following characteristics:
(a) negative staining by indirect immunofluorescence microscopy for GCTM-2, TRA1-60, TG343 and SSEA-4;
(b) positive staining for TG30 and antibody CAM5.2 against low molecular weight cytokeratins;
(c) low intensity fluorescence for the intermediate filament marker vimentin;
(d) expression of tetraspannin cell surface marker CD9;
(e) no positive fluorescence for SSEA-1, PHM4 (HLA antigen) and the epithelial marker EpCAM;
(f) expression of genes known to be transcriptionally active within pluripotent stem cell populations including Oct-4, Cripto, Pax6 and Genesis; or
(g) no transcripts for the endogerm genes alphafetoprotein, vitronectin, transferrin, or nestin.

49. An SCL-cell derived from the sub-population of cells according to claim 48.

50. An SCL cell according to claim 49 having the ability to differentiate in vitro to form neurospheres, neural cells, endothelial cells or extraembryonic endoderm by spontaneous differentiation.

51. A method of isolating a stem cell-like (SCL) cell which is morphologically distinct from HES cells and has stem cell-like (SCL) characteristics, said SCL cell having the ability to develop into various cell types, said method comprising:
obtaining a differentiating HES cell sub-population comprising spontaneously differentiated HES cells;
identifying SCL cells by negative expression of cell surface markers characteristic of SCL cells including GCTM-2, TRA 1-60, TG343 and SSEA-4 and positive expression of TG 30 or CD9₁₃ antigen; and
isolating said SCL cells expressing said negative and positive expression.

52. A method of isolating a stem cell-like (SCL) cell which is morphologically distinct from HES cells and has stem cell-like (SCL) characteristics, said SCL cell having the ability to develop into various cell types, said method comprising:
obtaining a differentiating HES cell sub-population according to any one of claims 26 to 28 comprising spontaneously differentiated HES cells;
identifying SCL cells by negative expression of cell surface markers characteristic of SCL cells including GCTM-2, TRA 1-60, TG343 and SSEA-4 and positive expression of TG 30 antigen or CD9 antigen; and
isolating said SCL cells expressing said negative and positive expression.

53. A method according to claim 52 wherein the SCL cells are further identified by at least on of the following characteristics:
(a) negative staining by indirect immunofluorescence microscopy for GCTM-2, TRA1-60, TG343 and SSEA-4;
(b) positive staining for TG30 and antibody CAM5.2 against low molecular weight cytokeratins;
(c) low intensity fluorescence for the intermediate filament marker vimentin;
(d) expression of tetraspannin cell surface marker CD9;
(e) no positive fluorescence for SSEA-1, PHM4 (HLA antigen) and the epithelial marker EpCAM;
(f) expression of genes known to be transcriptionally active within pluripotent stem cell populations including Oct-4, Cripto, Pax6 and Genesis; or
(g) no transcripts for the endogerm genes alphafetoprotein, vitronectin, transferrin, or nestin.

54. An SCL cell derived from a method according to claim 52.

55. An isolated and differentiated epithelial stem cell derived from a HES cell culture.

56. An isolated and differentiated epithelial stem cell derived from a subpopulation of HES cells according to any one of claims 26 to 28.

57. An isolated and differentiated epithelial stem cell according to claim 56 which is an endoderm progenitor cell.

58. An isolated and differentiated epithelial stem cell according to claim 56 wherein said cells show any one of the following characteristics:
(a) positive staining for EpCam and/or low molecular weight cytokeratins, albumin and GTCM-5;
(b) negative staining for GCTM-2;
(c) expression of the endoderm marker alphafetoprotein and/or the gut cell specific A33 antibody;
(d) no staining for the markers selected from the group including PHM4 (HLA Class I antigen), CD34 or the stem cell marker Oct-4;
(e) expression of transcripts for, Sox-17, and α-1 anti-trypsin;
(f) expression of hepatocyte nuclear factor (HNF)-3α, and HNF-4 or Pax 6; or
(g) no gene expression of Oct-4 or CR-1.

59. A differentiated epithelial stem cell according to claim 56 which is responsive to cholera toxin and requires STO cells.

60. A differentiated epithelial stem cell according to claim 56 which differentiates into a cell selected from the group including gut, liver, pancreas or lung cells.

61. A method of isolating a differentiated epithelial stem cell, said cell having the ability to develop into various cell types, said method comprising:
obtaining a differentiating HES cell sub-population comprising differentiating HES cells;
identifying epithelial stem cells morphologically and by expression of cell surface markers; and
isolating said cells and culturing the cells on a STO mouse fibroblast layer in a HES media including cholera toxin.

62. A method according to claim 61 further including sub-culturing the differentiating HES sub-population in the presence of a growth medium comprising DMEM, glucose, sodium pyruvate, nicotinamide, and foetal calf serum.

63. A method of isolating an epithelial stem cell, said cell having the ability to develop into various cell types, said method comprising:
obtaining a differentiating HES cell sub-population according to any one of claims 26 to 28 comprising differentiating HES cells;
identifying epithelial stem cells morphologically and by expression of cell surface markers; and
isolating said cells and culturing the cells on a STO mouse fibroblast layer in a HES media including cholera toxin.

64. A method according to claim 61 or 62 wherein the epithelial cells are identified by having at least one of the following characteristics:
(a) positive staining for EpCam and/or low molecular weight cytokeratins, albumin and GTCM-5;
(b) negative staining for GCTM-2;
(c) expression of the endoderm marker alphafetoprotein and/or the gut cell specific A33 antibody;
(d) no staining for the markers selected from the group including PHM4 (HLA Class I antigen), CD34 or the stem cell marker Oct-4;
(e) expression of transcripts for Sox-17, and α-1 anti-trypsin;
(f) expression of hepatocyte nuclear factor (HNF)-3α, and HNF-4 or Pax 6; or
(g) no gene expression of Oct-4 or CR-1.

65. An epithelial cell prepared by the method according to claims 61 or 62.

66. An epithelial cell according to claim 65 which is a hepatocyte.

67. A sub-population of HES cells having the ability to develop into various cell types and isolated by a method comprising:
obtaining a source of HES cells;
identifying HES cells that are at least GCTM-2 positive; and
selecting for or against those cells which are GCTM-2 positive;
separating the sub-populations of cells that are and are not GCTM-2 positive; and
sub-culturing the sub-populations of HES cells.

68. A sub-population of HES cells according to claim 67 which is an SCL cell or an epithelial stem cell.
